(19) United States
(12) Patent Application Publication
(10) Pub. No.: US 2024/0391985 A1
(43) Pub. Date: Nov. 28, 2024

(54) NKD2 AS TARGET FOR TREATING RENAL FIBROSIS

(71) Applicant: RHEINISCH-WESTFÄLISCHE TECH-NISCHE HOCHSCHULE (RWTH) AACHEN, Aachen (DE)

(72) Inventor: Rafael Johannes Thomas KRAMANN, Aachen (DE)

(21) Appl. No.: 18/249,694

(22) PCT Filed: Oct. 28, 2021
(86) PCT No.: PCT/EP2021/080068

§ 371 Date: Apr. 19, 2023

(30) Foreign Application Priority Data
Oct. 30, 2020 (DE) 10 2020 128 677.5

(51) Int. Cl. C07K 16/18; A61P 13/12; C12N 15/113; G01N 33/68

(52) U.S. Cl. CPC C07K 16/18; A61P 13/12; C12N 15/113; G01N 33/6845; C12N 2310/11; C12N 2310/14; G01N 2500/20

(57) ABSTRACT

The present application relates to the identification of the role of naked cuticle homolog 2 (Nkd2) gene-derived protein in the development of chronic kidney disease, in particular of progressive chronic kidney disease and kidney fibrosis. The present invention particularly relates to methods for identifying compounds that bind to Nkd2 protein, and to the use of Nkd2 for screening and identifying Nkd2-interacting compounds. The invention further relates to pharmaceutical compositions for use in the treatment of kidney diseases, in particular to pharmaceutical compositions comprising agents binding to and/or inhibiting NKD2 protein.

### FIELD OF THE INVENTION

The present invention relates to the role of naked cuticle homolog 2 (Nkd2) protein in the development of chronic kidney disease, in particular of progressive chronic kidney disease and kidney fibrosis. The present invention particularly relates to methods for identifying compounds that bind to Nkd2 protein, and to the use of Nkd2 for screening and identifying Nkd2-interacting compounds. The invention further relates to pharmaceutical compositions for use in the treatment of kidney diseases, in particular to pharmaceutical compositions comprising agents binding to and/or inhibiting Nkd2 protein.

### BACKGROUND

Chronic kidney disease (CKD) affects more than 10% of the world population, and its prevalence is increasing. Independent of the initial type of injury, the final common pathway of kidney injury is kidney fibrosis. Kidney fibrosis is the hallmark of chronic kidney disease progression, however, currently no antifibrotic therapies exist. The degree of kidney fibrosis is inextricably linked to loss of kidney function and clinical outcomes in CKD, and kidney fibrosis is therefore considered a key therapeutic target in CKD. No approved therapies exist for the treatment of kidney fibrosis, and this is largely because the cellular origin, functional heterogeneity and regulation of scar-producing cells in the human kidney remain unclear, and continue to represent a major source of debate in the field (Duffield 2014; Falke et al. 2015). The only treatment options are continuous renal replacement therapy (dialysis) and kidney transplantation. Both options are associated with high personal inconvenience for the patient and also represent high economic burden for national health systems. Therefore, novel therapeutic approaches are highly desirable.

Kidney fibrosis is defined by excessive deposition of extracellular matrix, which disrupts and replaces the functional parenchyma that leads to organ failure. Kidney's histological structure can be divided into three main compartments, all of which can be affected by fibrosis, specifically termed glomerulosclerosis in glomeruli, interstitial fibrosis in tubulointerstitium and arteriosclerosis and perivascular fibrosis in vasculature (Djudjai and Boor 2019).

Kidney fibrosis is characterized by high expression, secretion and accumulation of extra-cellular matrix (ECM) proteins like collagen-1. Myofibroblasts represent the major source of ECM during kidney fibrosis, and their cellular origin continues to be controversial (Duffield 2014; Friedman et al 2013; Kriz et al 2011; Kramann and DiRocco 2013). Single cell RNA sequencing and mapping allows the dissection of cellular heterogeneity of complex tissues and disease processes, and generates novel insights into disease-mediating cell populations and mechanisms (Ramachandran et al 2019; Dobie and Henderson 2019). In the past, genetic fate tracing data in mice, that had been extended by various staining approaches in human tissue, had suggested that a wide range of different cell types such as epithelial, endothelial, circulating hematopoietic cells as well as resident mesenchymal cells contribute to fibrosis (Duffield 2014; Friedman et al 2013; Kramann and DiRocco 2013).

Thus, the problem underlying the present invention is the provision of methods and means for identifying agents, compounds and compositions, as well as said agents, compounds and compositions, for use in the treatment of chronic kidney disease.

### SUMMARY OF THE INVENTION

The present invention provides methods and means for identifying agents, compounds and compositions for use in the treatment of chronic kidney disease, in particular for identifying highly effective agents, compounds and compositions for use in the treatment of progressive chronic kidney disease and kidney fibrosis.

As disclosed herein, the inventors found that Naked Cuticle Homolog 2 (NKD2) protein is produced in terminally differentiated myofibroblasts which are involved in kidney fibrosis, but not in cells which are expressing marker proteins for pericytes and fibroblasts and only small amounts of extracellular matrix protein. It was further found that Nkd2-expressing cells have an increased activity of pro-fibrotic signal transduction pathways. The inventors further found that reduction of fibrosis can be achieved by deletion of Nkd2 gene or knock-down of Nkd2 expression, thus identifying said gene as relevant for the production of extracellular matrix and fibrosis. Hence, the inventors for the first time identified Nkd2 as a new target and thus a new therapeutic approach for the development of therapeutic agents which inhibit Nkd2 gene expression and/or NKD2 protein activity by use of small-molecule agents (Smols), peptides or biologics.

In view of the prior art, it was hence one object of the present invention to provide a method for reducing extracellular matrix (ECM) protein expression and/or secretion by a given cell.

It was one further object of the present invention to provide a method for the identification of an agent that binds to and/or inhibits Naked Cuticle Homolog 2 (NKD2) protein, or a fragment thereof.

It was one further object of the present invention to provide a method of using a nucleic acid encoding the naked cuticle homolog 2, or a fragment thereof, or the Naked Cuticle Homolog 2 (NKD2) protein, or a fragment thereof, for the identification of an agent binding to NKD2, or a fragment thereof.

It was one further object of the present invention to provide agents for use in the treatment of chronic kidney disease, in particular for use in the treatment of progressive chronic kidney disease and/or kidney fibrosis, based on the findings described above.

It was one further object of the present invention to provide pharmaceutical compositions comprising said agents, and methods for producing such pharmaceutical compositions, based on the findings described above.

These and further objects are met with methods and means according to the independent claims of the present invention. The dependent claims are related to specific embodiments.

The invention and general advantages of its features will be discussed in detail below.

### DESCRIPTION OF THE FIGURES

FIG. 1. a. A schematic of the nephron structure and the cell types in different niches. b. A UMAP embedding of 51,849 MME-(CD10-) single cell transcriptomes from 15 human kidneys. Colors refer to five major cell types: epithelial (n=9,280), endothelial (n=29,814), immune (n=9,616), mesenchymal (n=3,115) and neuronal (Schwann cells, n=24). For information on abbreviations see 1d. c. A correlation network representation of the single cell clustering results. Nodes represent cell clusters. Edges (line connections) represent correlations between clusters. Network layout was determined using the force directed layout implemented in ggraph R package (https://cran.r-project.org/web/packages/ggraph/index.html). d. Scaled gene expression of the top 10 specific genes in each cell type/state cluster. Gene ranking per cluster was obtained using genesorteR. Cell cluster labels refer to a grouping of cell types/states into 29 canonical cell types (B Cells (B): n=3,101, T Cells (T): n=484, Natural Killer Cells (NK): n=740, Plasma Cells (P): n=167, Mast Cells (Mast): n=142, Dendritic Cells (DC): n=841, Monocytes (Mono): n=1,111, Macrophages 1 (Mac1): n=1,476, Macrophages 2 (Mac2): n=615, Macrophages 3 (Mac3): n=939, Arteriolar Endothelium (Art1): n=901, Glomerular Capillaries (GC): n=4377, venular Endothelium (Ven): n=2724, Lymph Endothelium (1En): n=509, Vasa Recta 1 (VR1): n=5,355, Vasa Recta 2 (VR2): n=2,023, Vasa Recta 3 (VR3): n=3,051, Vasa Recta 4 (VR4): n=726, Vasa Recta 5 (VR5): n=3,271, Vasa Recta 6 (VR6): n=4,378, Injured Endothelial Cells (iEn): n=2,499, Vascular Smooth Muscle Cells (VSMC): n=426, Pericytes 1 (Pe1): n=455, Pericytes 2 (Pe2): n=188, Fibroblasts 1 (Fib1): n=761, Fibroblasts 2 (Fib2): n=208, Fibroblasts 3 (Fib3): n=246, Myofibroblasts 1a (MF1a): n=525, Myofibroblasts 1b (MF1b): n=306, Proximal Tubule (PT): n=917, Injured Proximal Tubule (iPT): n=909, Descending Thin Limb (DTL: n=806, Connecting Tubule (CNT): n=662, Macula Densa Cells (Mdc): n=869, Thick Ascending Limb 2 (TAL2): n=692, Thick Ascending Limb 3 (TAL3): n=315, Thick Ascending Limb 4 (TAL4): n=390, Intercalated Cells 3 (IC3): n=62, Intercalated Cells 4 (IC4): n=78, Intercalated Cells 5 (IC5): n=33, Intercalated Cells 6 (IC6): n=39, Intercalated Cells 7 (IC7): n=40, Intercalated Cells 9 (IC9): n=72, Intercalated Cells A (IC-A): n=754, Intercalated Cells B (IC-B), n=316, Urothelial Cells (Ure): n=246, Podocytes (Pod): n=44, Schwann Cells: n=24). Cell clusters are in columns, genes are in rows. Each column is the average expression of all cells in a cluster. In FIG. 1d_1 expression of genes is depicted which are overexpressed. In FIG. 1d_2 expression of genes is depicted which are underexpressed/expressed at a lower level. e. Stratification of single cells according to patient clinical parameters (CKD-Chronic Kidney Disease, eGFR=estimated Glomerular Filtration Rate). f. A UMAP embedding of 31,875 CD10+ (CD10+) single cell transcriptomes stratified according to the patient clinical parameters. g. Log fold change of cell cycle stage assignment frequencies in healthy and CKD epithelial cells relative to a random model of frequencies. Positive numbers represent enrichment, negative numbers represent depletion. h. KEGG pathway enrichment for CD10+ cells. i. Cells from patients with chronic kidney disease are enriched for cells with a high ECM expression (ECM=Extracellular Matrix, CD10-cells). j. Violin plots of cells' ECM score stratified according to major cell types and Healthy/CKD in CD10-cells. P-value of differences in eGFR categories: Mesenchymal (p<0.001), Immune (p<0.001), Epithelial (p<0.001), Endothelial (p<0.001). k. Violin plots of cells ECM score for cells identified as Mesenchymal, stratified by major cell types and by Healthy/CKD. P-value of differences in eGFR categories: Fib1 (0.0001), Fib2 (n.s.), Fib3 (n.s.), MF1a (n.s.), MF1b (n.s.), Pe1 (n.s.), Pe2 (n.s.), SMC (n.s.) l. Number of cells per mesenchymal cell type and clinical parameter. m. A UMAP embedding of Fibroblast/Pericyte/Myofibroblast cells from 13 human kidneys (n=2,689). The different cell types are separated by dashed lines. Continuous lines refer to a lineage tree predicted by slingshot. n. Expression of selected genes shown in a UMAP of Figure b. o. A diffusion map embedding of pericytes, fibroblasts and myofibroblasts and the expression of Col1a1 on the same embedding.

FIG. 2. a. A UMAP embedding of 37,800 PDGFRb+ single cell transcriptomes from 8 human kidneys. The four major cell types are separated by dashed lines: epithelial (n=461), endothelial (n=2,341), immune (n=20,838), mesenchymal cells (n=25,385). Cell types/states were identified by unsupervised clustering of single cell transcriptomes (see Methods): fibroblasts 1 (Fib1), fibroblasts 2 (Fib2), fibroblasts 3 (Fib3), pericytes (Pe), vascular smooth muscle cells (VSMCs), mesangial cells (Mesa), myofibroblasts 1 (MF1), myofibroblasts 2 (MF2), myofibroblasts 3 (MF3), mesangial cells (Mesa), macrophage 1 (MC1), macrophage 2 (MC2), dendritic cells (DC), arteriolar endothelial cells (Art), glomerular endothelial cells (GC), vasa recta (VR), injured endothelium (iEn), proximal tubule (PT), injured proximal tubule (iPT), intercalated cells (IC), collecting duct principal cells (PC), thick ascending limb (TAL) b. Stratification of single cells according to patient clinical parameters (CKD=chronic kidney disease, eGFR=estimated glomerular filtration rate). c. Expression of selected genes shown as UMAP from Figure a. d. Scaled gene expression of the top 10 genes in each cell type/state cluster. Gene ranking per cell cluster was determined by genesorteR. Cell cluster labels refer to the cell clusters highlighted in a. and b. Cells are in columns (100 cells/column each), genes are in rows. In FIG. 2d_1 expression of genes is depicted which are overexpressed. In FIG. 2d_2 expression of genes is depicted which are underexpressed/expressed at a lower level. e. A Diffusion Map embedding of PDGFRb+ fibroblast/myofibroblast/pericyte cells (n=23,883) and the expression of selected genes on the same embedding. Lines correspond to the three lineages (lineage L1, L2 and L3) predicted by Slingshot. f. Representative image of multiplex RNA-in-situ hybridization for Meg3, Notch3, Postn in n=35 human kidneys. Scale bar left 10 μm, right 25 μm. From top to down: image of RNA in situ hybridization for Meg3, Notch, Postn and Dapi/only detection of Postn/Postn plus Notch-3/Postn plus Meg3/PostIn plus DAPI. Quantification of Meg3/Notch3 double positive cells. g. Top left: Gene expression dynamics for overexpression along pseudotime axis for Lineage 1 (Pericyte to Myofibroblast, see e.). Cells (in columns) were ordered along pseudotime axis, and genes (in rows) that correlate with pseudotime were selected and plotted along pseudotime (see Methods). Each 10 cells were averaged in one column. Genes were grouped in seven groups signifying their pseudotime expression pattern. Selected example genes are indicated. Bottom left: Image according to top left, but underexpression/expression at low levels is depicted. Top right: Cell cycle stage along pseudotime as percent of each 2000 cells along pseudotime. Bottom right: PID Signaling pathway enrichment analysis along pseudotime.

FIG. 3. a. Fate tracing experiment design (top) and visualization of PDGFRbCreER-tdTomato (bottom) in UUO (unilateral ureteral obstruction) mouse kidney model compared to sham surgery. Top: Detection of PDGFRb-tdtom and DAPI; Middle: Detection of PDGFRb-tdtom; Bottom: Detection of DAPI. b. Representative image of Col1a1 in-situ hybridization in a PDGFRbCreER; tdTomato kidney after UUO surgery. From top to bottom: Detection of PDGFRb-tdtom+Col1+DAPI/Detection of Col1/Detection of PDGFRb-tdtom+Col1/Detection of Col1+DAPI. c. Percentage of Col1a1-mRNA expressing cells that co-express tdTomato at day 10 after UUO surgery (n=3). d. Time-course UUO experiment design. UUO was performed in PDGRb-eGFP mice and eGFP positive single cells from mouse kidneys were isolated at days 0, 2 and 10 after UUO induction and assayed using Smart-Seq v2. e. A UMAP embedding of the cells collected in the time-course UUO experiment depicted in d. Cell types were identified by unsupervised clustering (see Methods) (parietal epithelial cells (PECs): n=68, matrix producing cells (MP): n=76, injured smooth muscle cells 1 (iSMCs1): n=112, injured smooth muscle cells 2 (iSMCs2): n=77, injured smooth muscle cells 3 (iSMCs3): n=76, mesangial cells (Mesa): n=74, pericytes 1 (Pe1): n=113, renin producing smooth muscle cells (rSMC): n=101, smooth muscle cells 1 (SMC1): n=172, pericytes 2 (Pe2): n=83). f. Percent of cells occurring in each cell type per time-point. Each column sums to 100. g. Expression of selected genes in all 10 cell clusters. h. Expression of selected genes on the UMAP embedding from e. i. Immuno-fluorescence (IF) staining in sham and UUO (day 10) mouse kidney showing Pdgfra expression in a subset of PDGFRbCreER; tdTomato positive cells (arrows). From left to right: Detection of PDGFRbCreERtdTomato+PDGFRa+DAPI/Detection of PDGFRbCreERtdTomato/Detection of PDGFRa/Detection of DAPI. j. RNA in-situ hybridization showing co-localization of Col1a1 expression in PDGFRa/PDGFRb double-positive cells. Col1a1/PDFGRa/PDFRb triple-positive cells (arrows) occur solely in the kidney interstitium. From top to bottom: Detection of PDGFRa+Col1a1+PDGFRb+DAPI/Detection of PDGFR-a/Detection of PDGFRa+Col1a1/Detection of PDGFRa+PDGFRb/Detection of PDGFRa+DAPI. k. Left: Col1a1 expression and ECM score in CD10 negative cells (FIG. 1b-c) stratified according to PDGFRa and PDGFRb expression. Right: Percent of Col1a1 positive and negative cells in the same data set, stratified as described above. Col1a1 negative cells are detectable in PDGFRa/b double-negative cells, while Col1a1 positive cells occur predominantly in PDGFRa/b double-positive cells. Group comparisons: (other genes) vs. (a/b): p<0.001, (a-) vs. (a/b): p<0.001, (b) vs. (a/b): p<0.001, (other genes) vs. (a): p<0.001, (b) vs. (a): p<0.001, (other genes) vs. (b): p<0.001. Bonferroni corrected p-values based on Wilcoxon rank sum test. l. Distribution of IF/TA-Score over 62 patients and representative image of a trichrome stained human kidney tissue microarray (TMA) stained by multiplex RNA in-situ hybridization using PDGFRa, PDGFRb and Col1a1 probes with nuclear counterstain (DAPI) of 62 kidneys (left), average scaled Col1a1 expression in the in-situ hybridization data stratified by PDGFRa/PDGFRb detection in the same data set (middle) and percent of Col1a1 positive and negative cells in the same data set stratified as above (right). Group comparison: (a/b) vs. (col1a1): p<0.001, (a/b) vs. (b): p<0.001, (a/-) vs. (a): p<0.001. Bonferroni corrected p-values based on Wilcoxon rank sum test. Scale bars: in a 1000 μm, in b 10 μm, in i+j 50 μm, in k 10 μm. Multiplex RNA in situ hybridization from top to bottom: Detection of Col1a1+PDGFRa+PDGFRb+DAPI/Detection Col1a1/Detection of Col1+PDGFRa/Detection of Col1a1+PDGFRb/Nachweis col1+DAPI.

FIG. 4. a. Scheme of the UUO experiment. UUO was performed in PDGRb-eGFP mice. eGFP+/PDGFRb+ single cells were isolated from mouse kidneys and were assayed using 10× Genomics drop-seq at days 0 and 10 after UUO (n=5 each). b. Flow cytometric quantification of PDGFRa, PDGFRb and PDGFRa/b expressing cells at day 10 after UUO surgery compared to sham surgery. *p<0.05; **p<0.01 by one way ANOVA test with post-hoc Bonferroni correction. c. Left: A UMAP embedding of the cells isolated in the UUO experiment (depicted in a). (n=7,245). 4 major cell clusters could be identified (epithelial (n=223), endothelial (n=370), immune (n=199), mesenchymal cells (n=6,633). 10 cell types obtained by unsupervised clustering of single cell transcriptomes could be distinguished. Fibroblasts 1 (Fib1), myofibroblasts 1 (MF1), myofibroblasts 2 (MF2), myofibroblasts 3 (MF3), endothelial cells (EC), injured proximal tubular cells (iPT), unknown mesenchymal cells (uM), macrophages/monocytes (MC). Right: Percent of cells in each cell cluster in the sham or UUO mice. d. Expression of selected genes in each of the cell clusters (indicated in c). e. Image of extracellular matrix score (ECM score) visualized on the UMAP embedding from c. f. A violin plot of Col15a1 expression in the different cell clusters. Only mesenchymal cells are shown. g. A violin plot of collagen score in the different cell clusters. Only mesenchymal cells are shown. Collagen score is the average expression of core collagen genes as provided by Naba et al. h. Representative image of multiplex RNA in-situ hybridization for PDGFRa, PDGFRb and Meg3 in n=34 human kidneys. Meg3 colocalizes with PDGFRa and PDGFRb. From top to bottom: Detection of Meg3+PDGFRa+PDGFRb+DAPI/Detection of Meg3/Detection of Meg3+PDGFRa/Detection of Meg3+PDGFRb/Detection of Meg3+DAPI. i. Percent of Meg3-positive cells out of PDGFRa/b double-positive cells, quantified from RNA in-situ hybridization. j. A UMAP (left) and diffusion map (right) embeddings of fibroblast and myofibroblast cells (n=6,557). Cell clusters as indicated in c. Black lines indicate the lineage tree predicted by Slingshot. Bottom: Expression of select genes visualized on the same UMAP embedding. k. Signaling pathway enrichment in the same mesenchymal cell clusters.

FIG. 5. a. Expression of Nkd2 visualized on the UMAP embedding from FIG. 4c. (mouse Pdgfra/b double-positive cells). b. Percent of Col1a1 positive and negative cells in the same data set as a., stratified by Pdgfra and Nkd2 expression. Col1a1 negative cells occur mostly in PDGFRa/Nkd2 double-negative cells, while Col1a1 positive cells are most frequently also PDGFRa/Nkd2 double-positive cells. c. Scaled gene expression of genes identified as correlating (FIG. 5c_1) or anti-correlating (FIG. 5c_2) with Nkd2 expression in human PDGFRb-cells, depicted in FIG. 2 a-c. d. Representative image of multiplex RNA in-situ hybridization of PDGFRa, PDGFRb and NKD2 in n=36 human kidneys. From top to bottom: Detection of NKD2+PDGFRa+PDGFRb+DAPI/Detection of NKD2/Detection of NKD2+PDGFRa/Detection of NKD2+PDGFRb/Detection of NKD2+DAPI. e. Percent of NKD2+ cells out of PDGFRa/PDGFRb double-positive cells, quantified from RNA in-situ hybridization from patients with low or high interstitial fibrosis as blinded scored by a nephropathologist. f. Western blot verification of lentiviral Nkd2 overexpression. A HA tag is attached to the exogenous overexpressed protein. g. Expression of Col1a1, Fibronectin (Fn) and Acta2 (aSMA) quantified by qPCR after Nkd2 overexpression in human immortalized PDGFRb+ cells treated with transforming growth factor beta (TGFb) or vehicle (PBS). h. Verification of Nkd2 knock-out by Western-blot in multiple single cell clones (1,2,3) compared to non targeting gRNA clones (NTG). Nkd2 protein expression is completely deleted in Clone 2 due to a large insertion, while clones 1, 3 showed only reduced Nkd2 protein expression due to smaller indel mutations. i. Expression of Col1a1, Fibronectin (Fn) and Acta2 by RNA qPCR after Nkd2 knock-out in the same clones depicted in g. j. GSEA (Gene set enrichment analysis) of ECM genes in Nkd2-perturbed PDGFRb-kidney cells. “Shallow” was detected in clones 1+3 where NKD2 protein was still detected. “Severe” was detected in clone 2. k. Modification in strength of PID signaling pathway in PDGFRb+NKD2-KO clones and overexpression (up indicates up-regulated genes under indicated condition, and down indicates down-regulated genes) l. Representative image of multiplex RNA in-situ hybridization of PDGFRa, PDGFRb and NKD2 in human iPSC derived kidney organoids. From top to bottom: Detection of Nkd2+PDGFRa+Col1a1+DAPI/Detection of Nkd2/Detection of Nkd2+PDGFRa/Detection of Nkd2+Col1a1/Detection of Nkd2+DAPI. m. Quantification of fluorescent intensity of NKD2 in kidney organoids. n. Immunofluorescence staining of Col1a1 (black) in iPSC-derived kidney organoids. o. Quantification of collagen content in kidney organoids. ##p<0.01, ####p<0.0001 1 way ANOVA followed by Bonferroni' post-hoc test (vs. control NTG). * P<0.05, **p<0.01, and ***p<0.001, ****p<0.0001 by t test (e+m) or 1-way ANOVA followed by Bonferroni' post-hoc test (g, i vs. TGFb NTG)). Data represent the mean±SD. Scale bar: in c 10 μm, in 1+n 50 μm.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions.

According to a first aspect of the present invention, the present invention relates to a method for reducing extracellular matrix (ECM) protein expression and/or secretion by a given cell, wherein the method comprises at least one step selected from the group consisting of
(i) inhibiting or reducing nkd2 gene expression in said cell,
(ii) promoting degradation of NKD2 protein in said cell, and/or
(iii) inhibiting or reducing NKD2 protein activity in said cell.

Said inhibition or reduction of nkd2 gene expression can be achieved, for example, by nkd2 gene knock-down, knock-out, conditional gene knock-out, gene alteration, RNA interference, siRNA and/or antisense RNA. The inhibition or reduction of nkd2 gene expression can be achieved, for example, by antisense molecules such as antisense oligonucleotides, antisense conjugates, or catalytic nucleic acid molecules such as ribozymes. Such molecules can be produced in the cell by means of expression vectors, or can be introduced from outside of the cell.

The antisense oligonucleotides can be chemically modified in order to increase their stability and/or binding affinity. The chemical modification of the backbone chemistry of antisense oligonucleotides, for example, by phosphorothioate, phosphorodithioate, phosphoroamidite, alkyl-phosphotriester or boranophosphate was described in the prior art (for example, in WO00/49034A1).

Said promoting degradation of NKD2 protein in said cell can be achieved, for example, by proteases or other proteolytic molecules. Such proteases or proteolytic molecules can be heterologously synthesized by means of expression vectors in the cell, or can be synthesized in increased amounts in the cell by increasing homolog gene expression of protease-encoding genes in the cell, or can be introduced into the cell from outside of the cell.

Said inhibition or reduction of NKD2 protein activity can be achieved by use of an agent that binds to Naked Cuticle Homolog 2 (NKD2) protein.

Preferably, said given cell is a kidney cell, more preferably a kidney myofibroblast cell, most preferably a terminally differentiated kidney myofibroblast cell.

NKD2 protein has been shown to be a WNT antagonist. The Naked Cuticle (NKD) family includes Drosophila naked cuticle and its two vertebrate orthologs, naked cuticle homolog 1 (NKD1) and 2 (NKD2). The Nkd2 gene locus is located in chromosome 5p15.3. Loss of heterozygosity has been frequently found in these regions in different types of tumors, including breast cancer. Both NKD1 and NKD2 have been reported to antagonize canonical Wnt signaling by interacting with Dishevelled through their EF-hand-like motifs (Hu et al 2006). In addition, NKD2 has been demonstrated to bind to Dishevelled through its TGFα binding region (Li et al 2004). Human NKD1 and 2 are only 40% identical to each other and they are approximately 70% identical to their respective orthologs in mouse.

The C-terminus of NKD2 is highly disordered, while the N-terminal region of NKD2 contains most of the functional domain, which includes myristoylation, an EF-hand motif, a Dishevelled binding region, and a vesicle recognition and membrane targeting motif (Li et al 2004; Rousset et al 2001; Zeng et al 2000). NKD2 has been suggested to function as a switch protein through its several functional motifs (Hu et al 2006). The promoter region of NKD2 is hypermethylated in glioblastoma cells.

As described herein, the inventors of the present application identified NKD2 as a therapeutic target for the treatment of kidney fibrosis. Nkd2 has been found to be exclusively expressed in terminally differentiated PDGFRa+/PDGFRb+ myofibroblasts, which show high expression levels of the extracellular matrix protein collagen-1. This and other matrix proteins are produced by myofibroblasts, which predominantly arise by differentiation from fibroblasts and pericytes. More than 40% of all collagen-1-producing cells have been shown to be Nkd2/PDGFRa+. Cells expressing marker proteins for pericytes and fibroblasts, and secreting only low levels of matrix proteins, are lacking Nkd2 expression. In addition, in Nkd2-expressing myofibroblasts, increased activity of pro-fibrotic signal transduction pathways, such as TGF-β-, Wnt- and TNFα signal transduction pathways, has been detected. In Nkd2 overexpression and depletion experiments, respectively, it has been demonstrated that Nkd2 is relevant for the production of extracellular matrix proteins. Lentiviral over-expression of Nkd2 in human fibroblasts resulted in increased expression of pro-fibrotic matrix proteins like collagen-1 and fibronectin after stimulation by the pro-fibrotic factor TGF-β. CRISPR/Cas9-mediated knockout of Nkd2 could be shown to lead to a significant reduction of expression of collagen-1, fibronectin and ACTA2. Knock-down of Nkd2 by means of siRNA in organoids comprising all compartments of the human kidney, in which fibrotic changes had been induced by stimulation with IL1-13, has been shown to result in reduced collagen-1 expression and fibrosis.

Said NKD2 protein can be a mammalian, non-primate, primate, and in particular a human NKD2 protein, a fragment thereof.

According to another aspect of the present invention, the present invention relates to a method for the identification of an agent that binds to Naked Cuticle Homolog 2 (NKD2) protein, or a fragment thereof.

Said method may comprise at least the steps of
(i) providing the NKD2 protein, or a fragment thereof,
(ii) adding at least one agent to be screened for binding to the NKD2 protein, or a fragment thereof, and
(iii) identifying the at least one agent that has bound to the NKD2 protein, or the fragment thereof.

Preferably, said agent to be screened and identified according to the present invention is an NKD2 inhibitor or antagonist.

Said agent according to the present invention can be selected from the group consisting of a small-molecule compound, a peptide, and a biologic.

In the context of the present invention the term “small-molecule compound”, “small-molecule” (“smol”) or “chemical drug” relates to a low molecular weight (<1,000 daltons) organic compound, often with a size on the order of 1 nm. Many drugs are small molecules. Such small molecules may regulate a biological process. Small molecules may be able to inhibit a specific function of a protein. In the field of pharmacology the term “small molecule” particularly refers to molecules that bind specific biological macromulecules and act as an effector, altering the activity or function of a target. For example acetylsalicylic acid (ASA) is considered a small molecule drug, which measures 180 daltons and comprises 21 atoms. Such small molecule compounds often have little ability to initiate an immune response and remain relatively stable over time.

Said “biologic”, “biological drug”, “biologic therapeutic” or “biopharmaceutical” according to the present invention preferably is an antibody, or antigen-binding fragment thereof, or antigen-binding derivative thereof, or antibody-like protein, or an aptamer.

In a preferred embodiment of said method for the identification of an agent that binds to NKD2 protein, or a fragment thereof, said agent is member of a compound library.

Said compound library can comprise, e.g., small-molecule compounds, peptides, or biologic compounds, respectively.

In the context of the present invention, the term “(combinatorial) compound library” relates to collections of chemical compounds, small molecules, peptides or macromolecules such as proteins, in which multiple different combinations of related chemical, peptide or biologic species are comprised, which can be used together in particular screening assays or identification steps.

According to another aspect of the present invention, the present invention relates to the use of a nucleic acid encoding the naked cuticle homolog 2, or a fragment thereof, or the Naked Cuticle Homolog 2 (NKD2) protein, or a fragment thereof, in a method for the identification of an agent binding to NKD2, or a fragment thereof, as described above.

According to another aspect of the present invention, the present invention relates to an antibody, or antigen-binding fragment or derivative thereof, or antibody-like protein, that specifically binds to NKD2 protein.

Preferably, said antibody, or antigen-binding fragment or derivative thereof, or antibody-like protein, inhibits the NKD2 activity, i.e., acts as an inhibitor or antagonist of NKD2.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or V_{L}) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen binding portion” of the (monoclonal) antibody refers to one or more fragments of an antibody which retain the ability to specifically bind to the antigen in its native form. Examples of antigen binding portions of the antibody include a Fab fragment, a monovalent fragment consisting of the VL, VH, CL and CH1 domains, an F(ab′)₂ fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR).

The antibody, or antibody fragment or antibody derivative thereof, according to the present invention can be a monoclonal antibody. The antibody can be of the IgA, IgD, IgE, IgG or IgM isotype.

As used herein, the term “monoclonal antibody (mAb)” shall refer to an antibody composition having a homogenous antibody population, i.e., a homogeneous population consisting of a whole immunoglobulin, or a fragment or derivative thereof. Particularly preferred, such antibody is selected from the group consisting of IgG, IgD, IgE, IgA and/or IgM, or a fragment or derivative thereof.

As used herein, the term “fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs. All these items are explained below.

Other antibody derivatives known to the skilled person are Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, other scaffold protein formats comprising CDRs, and antibody conjugates.

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin proteins, and affilin proteins. Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

As used herein, the term “Fab” relates to an IgG fragment comprising the antigen binding region, said fragment being composed of one constant and one variable domain from each heavy and light chain of the antibody.

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually comprising serine(S) and/or glycine (G) residues. This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like.

IgG, scFv, Fab and/or F(ab)₂ are antibody formats which are well known to the skilled person. Related enabling techniques are available from respective textbooks.

According to preferred embodiments of the present invention, said antibody, or antigen-binding fragment thereof or antigen-binding derivative thereof, is a murine, a chimeric, a humanized or a human antibody, or antigen-binding fragment or antigen-binding derivative thereof, respectively.

Monoclonal antibodies (mAb) derived from mouse may cause unwanted immunological side-effects due to the fact that they contain a protein from another species which may elicit antibodies. In order to overcome this problem, antibody humanization and maturation methods have been designed to generate antibody molecules with minimal immunogenicity when applied to humans, while ideally still retaining specificity and affinity of the non-human parental antibody. Using these methods, e.g., the framework regions of a mouse mAb are replaced by corresponding human framework regions (so-called CDR grafting). WO200907861 discloses the generation of humanized forms of mouse antibodies by linking the CDR regions of non-human antibodies to human constant regions by recombinant DNA technology. U.S. Pat. No. 6,548,640 by Medical Research Council describes CDR grafting techniques, and U.S. Pat. No. 5,859,205 by Celltech describes the production of humanised antibodies.

As used herein, the term “humanized antibody” relates to an antibody, a fragment or a derivative thereof, in which at least a portion of the constant regions and/or the framework regions, and optionally a portion of CDR regions, of the antibody is derived from or adjusted to human immunoglobulin sequences.

According to another aspect of the present invention, the present invention relates to an agent obtained by the identification method as described above.

Said agent has the ability to specifically bind to Naked Cuticle Homolog 2 (NKD2) protein. In a preferred embodiment, said agent specifically binds with a high or particularly high affinity and/or avidity to NKD2 protein or a fragment thereof. In a preferred embodiment, said agent, when bound to NKD2, reduces or inhibits the NKD2 activity.

The term “specifically bind” as used herein means that said agent has a dissociation constant K_{D} to the NKD2 protein molecule or epitope thereof of at most about 100 μM. In an embodiment, K_{D} is about 100 μM or lower, about 50 μM or lower, about 30 μM or lower, about 20 μM or lower, about 10 μM or lower, about 5 μM or lower, about 1 μM or lower, about 900 nM or lower, about 800 nM or lower, about 700 nM or lower, about 600 nM or lower, about 500 nM or lower, about 400 nM or lower, about 300 nM or lower, about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, or about 10 nM or lower, about 1 nM or lower, about 900 pM or lower, about 800 pM or lower, about 700 pM or lower, about 600 pM or lower, about 500 pM or lower, about 400 pM or lower, about 300 pM or lower, about 200 pM or lower, about 100 pM or lower, about 90 pM or lower, about 80 pM or lower, about 70 pM or lower, about 60 pM or lower, about 50 pM or lower, about 40 pM or lower, about 30 pM or lower, about 20 pM or lower, or about 10 pM or lower, or about 1 pM or lower.

Said agent can serve for use in the treatment of chronic kidney disease, in particular wherein said chronic kidney disease is progressive chronic kidney disease and/or kidney fibrosis.

Said agent can be a small-molecule compound (smol), a peptide, or a biologic, preferably wherein said biologic is an antibody, or fragment thereof or derivative thereof, or antibody-like protein, or an aptamer.

The small-molecule compound according to the present invention may comprise, among other chemical backbones, substituents, groups or residues, for example, alkyl-, alkenyl-, alkinyl-, alkoxy-, aryl-, alkylene-, arylene-, amino-, halogen-, carboxylate derivate-, cycloalkyl-, carbonyl derivative-, heterocycloalkyl-, heteroaryl-, heteroarylen-, sulphonate-, sulphate-, phosphonate-, phosphate-, phosphine-, phosphinoxide groups.

According to another aspect of the present invention, the present invention relates to the use of an agent that binds to and/or inhibits Naked Cuticle Homolog 2 (NKD2) protein in a method of treating chronic kidney disease, preferably wherein the chronic kidney disease is progressive chronic kidney disease and/or kidney fibrosis. In a preferred embodiment, said agent, when bound to NKD2, inhibits the NKD2 activity.

The present invention relates to a method for treating or preventing chronic kidney disease, which method comprises administration, to a human or animal subject, of an agent that binds to and/or inhibits Naked Cuticle Homolog 2 (NKD2) protein in a therapeutically effective amount or dose.

As used herein, the term “effective amount” means a dose or an amount effective, at dosages and for periods of time necessary to achieve a desired result. Effective amounts may vary according to factors such as the disease state, age, sex and/or weight of the subject, the pharmaceutical formulation, the sub-type of disease being treated, and the like, but can nevertheless be routinely determined by one skilled in the art.

According to another aspect of the present invention, the present invention relates to a pharmaceutical composition comprising the antibody, or antigen-binding fragment or derivative thereof, or antibody-like protein, as described above, or the agent as described above, and optionally one or more pharmaceutically acceptable excipients. Preferably, said excipients can be selected from the group consisting of pharmaceutically acceptable buffers, surfactants, diluents, carriers, excipients, fillers, binders, lubricants, glidants, disintegrants, adsorbents, and/or preservatives.

According to another aspect of the present invention, the present invention relates to a method for the production of a pharmaceutical composition, comprising
(i) the method for the identification of an agent that binds to and/or inhibits NKD2 protein, or a fragment thereof, as described above, and furthermore
(ii) mixing the agent identified with a pharmaceutically acceptable carrier.

According to another aspect of the present invention, the present invention relates to a composition comprising a combination of (i) the antibody, or antigen-binding fragment or derivative thereof, or antibody-like protein, as described above, or the agent that binds to Naked Cuticle Homolog 2 (NKD2) protein as described above, or the pharmaceutical composition as described above, and (ii) one or more further therapeutically active compounds.

Said pharmaceutical composition may comprise one or more pharmaceutically acceptable buffers, surfactants, diluents, carriers, excipients, fillers, binders, lubricants, glidants, disintegrants, adsorbents, and/or preservatives.

Said pharmaceutical composition may be administered in the form of powder, tablets, pills, capsules, or pearls. In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

Said pharmaceutical formulation may further comprise one or more stabilizer, which may be, e.g., an amino acid, a sugar polyol, a disaccharide and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least oral, parenteral, intravenous, intramuscular or subcutaneous administration. Alternatively, said conjugate according to the present invention may be provided in a depot formulation which allows the sustained release of the active agent over a certain period of time.

In still another aspect of the present invention, a primary packaging, such as a prefilled syringe or pen, a vial, or an infusion bag is provided, which comprises said formulation according to the previous aspect of the invention.

The prefilled syringe or pen may contain the formulation either in lyophilised form (which has then to be solubilised, e.g., with water for injection, prior to administration), or in aqueous form. Said syringe or pen is often a disposable article for single use only, and may have a volume between 0.1 and 20 ml. However, the syringe or pen may also be a multi-use or multi-dose syringe or pen.

According to another aspect of the present invention, the present invention relates to a therapeutic kit of parts comprising:
(i) the pharmaceutical composition as described above,
(ii) a device for administering the composition, and
(iii) optionally, instructions for use.

### Sequences

TABLE 1
Human NKD2 amino acid sequence

| SEQ ID No. | Sequence | Description |
|---|---|---|
| SEQ ID No. 1 | MGKLQSKHAAAARKRRESPEGDSFVASAYASGRKGAEE | NKD2 |
| | AERRARDKQELPNGDPKEGPFREDQCPLQVALPAEKAE | protein, |
| | GREHPGQLLSADDGERAANREGPRGPGGQRLNIDALQC | human |
| | DVSVEEDDRQEWTFTLYDFDNCGKVTREDMSSLMHTIY | |
| | EVVDASVNHSSGSSKTLRVKLTVSPEPSSKRKEGPPAG | |
| | QDREPTRCRMEGELAEEPRVADRRLSAHVRRPSTDPQP | |
| | CSERGPYCVDENTERRNHYLDLAGIENYTSRFGPGSPP | |
| | VQAKQEPQGRASHLQARSRSQEPDTHAVHHRRSQVLVE | |
| | HVVPASEPAARALDTQPRPKGPEKQFLKSPKGSGKPPG | |
| | VPASSKSGKAFSYYLPAVLPPQAPQDGHHLPQPPPPPY | |
| | GHKRYRQKGREGHSPLKAPHAQPATVEHEVVRDLPPTP | |
| | AGEGYAVPVIQRHEHHHHHEHHHHHHHHHFHPS | |

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1: Materials and Methods

### Human Tissue Processing

Kidney tissues were sampled by the surgeon from normal and tumor regions. The tissue was snap-frozen on dry-ice or placed in prechilled University of Wisconsin solution (#BTLBUW, Bridge to Life Ltd., Columbia, U.S.) and transported to our laboratory on ice. Tissues were sliced into approximately 0.5-1 mm3 pieces and then transferred to a C-tube (Miltenyi Biotec) and processed on a gentle-MACS (Miltenyi Biotec) using the program spleen 4. The tissue was then digested for 30 min at 37° C. with agitation at 300 RPM in a digestion solution containing 25 μg/ml Liberase TL (Roche) and 50 μg/ml DNase (Sigma) in RPMI (Gibco). Following incubation, samples were processed again on a gentle-MACS (Miltenyi Biotec) using the same program. The resulting suspension was passed through a 70 μm cells strainer (Falcon), washed with 45 ml cold PBS and centrifuged for 5 minutes at 500 g at 4° C. Cells were counted using a hemocytometer with trypan blue staining. Live, single cells were enriched by FACS-sorting and gating on DAPI negative cells with further enrichment of epithelial cells by CD10 staining or PDGFRβ staining for fibroblast. On average it took 5-6 hours from obtaining biopsies to generating single cell suspensions.

### Mice

PDGFRBCreERt2 (i.e. B6-Cg-Gt (Pdgfrβ-cre/ERT2) 6096Rha/J, JAX Stock #029684) and Rosa26tdTomato (i.e. B6-Cg-Gt(ROSA)26Sorttm(CAG-tdTomato)Hze/J JAX Stock #007909) were purchased from Jackson Laboratories (Bar Harbor, ME, USA). Offspring were genotyped by PCR according to the protocol from the Jackson laboratory. Pdgfrb-BAC-eGFP reporter mice were developed by N. Heintz (The Rockefeller University) for the GENSAT project. Genotyping of all mice was performed by PCR. Mice were housed under specific pathogen-free conditions at the University of Edinburgh or RWTH Aachen. UUO was performed as previously described.2 Briefly, after flank incision, the left ureter was tied off at the level of the lower pole with two 7.0 ties (Ethicon). Mice were sacrificed on day 10 after the surgery. Animal experiment protocols were approved by the LANUV-NRW, Germany and by the UK Home Office Regulations. All animal experiments were carried out in accordance with their guidelines. PDGFRbeGFP male mice for SMART-Seq2 were used, born within 10 days of each other, and between 9 and 11 weeks old at the time of surgery and sacrificed as indicated. For inducible fate tracing PDGFRbCreER; tdTomato mice (8 weeks of age, 2 male/3 female) received tamoxifen 3 times via gavage (10 mg p.o.) followed by a washout period of 21 days and then subjected to UUO surgery or sham (as above) and sacrificed at 10 days after surgery.

### Single Cell Isolation in Mouse

Euthanized mice were perfused via the left heart with 20 ml NaCl 0.9% to remove blood residues from the vasculature. The kidneys were surgically removed, cut into small slices and placed in a 15 ml tube (Falcon) on ice-cold PBS containing 1% FCS. To isolate single kidney cells, a combination of enzymatic and mechanical disruption was used as described above for human single cell isolation. Overall the viability was over 80% using this method.

### FACS

Cells were labeled with the following monoclonal, directly fluorochrome conjugated antibodies: anti-CD10 human (clone HI10a, biolegend), anti-PDGFRb mouse (clone PR7212, R&D), anti-PDGFRalpha mouse (clone APA5, biolegend), anti-CD31 mouse (clone Meg13.3, biolegend), anti-CD45 mouse (clone 30_F11). Isolated cells were resuspended in 1% PBS-FBS on ice at a final concentration of 1×107 cells/ml. Cells were pre-incubated with Fc-Block (TruStainFx human, TruStainFx mouse Clone 91, biolegend) and then incubated with the above antibodies for 30 minutes on ice protected from light diluted 1:100 in 2% FBS/PBS. For human anti-PDGFRb staining goat anti-mouse Dyelight 405 (poly24091, biolegend) was used as secondary antibody. All compensation was performed at the time of acquisition using single color staining and negative staining and fluorescence minus one controls. The cells were sorted in the semi-purity mode targeting an efficiency of >80% with the SONY SH800 sorter (Sony Biotechnology; 100 μm nozzle sorting chip Sony). For plate based sorting for SMART-Seq, cell sorting was performed on a FACS Aria II machine (Becton Dickinson, Basel, Switzerland).

Single Cell Assays Incl. Smart-Seg2 and 10× Genomics 3′ Sc-RNA-Seg (V2 and V3)

For Smart-Seq2 single cells were processed by SciLifeLab—Eukaryotic Single cell Genomic Facility (Karolinska Institute). Before shipping single cells were sorted into wells of a 384-well plate containing pre-prepared lysis buffer. Libraries were sequenced on an Illumina HiSeq 4500. The single cell solution of cells and primary human kidney cells were run in parallel on a Chromium Single Cell Chip kit and library were performed using Chromium Single Cell 3′ library kit V2 and i7 Multiplex kit (PN-120236, PN-120237, PN-120262, 10× Genomics) according to the manufacturer's protocol. The library quality was determined using D1000 ScreenTape on 2200 TapeStation system (Agilent Technologies). Sequencing was performed on a Illumina Novaseq platform using S1 and S2 flow cells (Ilumina).

### Human Kidney Fibrosis Evaluation

PAS stained sections of the kidneys were analyzed and scored in a blinded fashion by an experienced nephropathologist. All sections were screened for specific kidney diseases, however, no indication of specific glomerular of tubulointerstitial or vascular diseases, apart from age-related changes or hypertensive nephropathy were observed. The extent of interstitial fibrosis and tubular atrophy were assessed as two separate parameters as % of affected cortical area. Extent of global glomerulosclerosis was estimated as % of globally sclerotic glomeruli from all glomeruli. Extent of arteriosclerosis, i.e. fibroelastic thickening of intima compared to thickness of media, was scored from 0-3, with 0—no, 1—mild (<50%), 2—medium (51-100%) and 3—severe (>100% thickened intima compared to media).

For collagen I and III immunohistochemistry on μm sections of formalin-fixed and paraffin-embedded renal tissues were processed for indirect immunoperoxidase, by removing paraffin by incubation in xylene (3×5 min) and subsequent rehydration with descending concentration of ethanol (3×2 minutes 100% ethanol, 2×2 minutes 95% ethanol, 1×2 minutes 70% ethanol). Endogenous peroxidase activity was blocked with 3% H2O2 in distilled water for 10 minutes at room temperature and washed (2× in PBS), followed by incubation with primary antibodies [Collagen I (Southern Biotech) Cat No. 1310-01; Collagen III (Southern Biotech) Cat No. 1330-01] in 1% BSA/PBS at room temperature for 1 hour in a humid chamber. Afterwards slides were washed two times for 5 minutes in PBS and biotinylated secondary antibody was added (30 minutes). Avidin-biotin complex was added and incubated for 30 minutes and then incubated in DAB-solution for 10 minutes at 37° C. The reaction was stopped by washing in H2O and slides were counterstained with methyl green for 4 minutes. Finally, slides were dehydrated in ascending ethanol and xylene. Using a whole slide scanner (NanoZoomer HT, Hamamatsu Photonics, Hamamatsu, Japan), fully digitalized images of immunohistochemically stained slides were further processed and analyzed using the viewing software NDP.view (Hamamatsu Photonics, Hamamatsu, Japan) and ImageJ (National Institutes of Health, Bethesda, MD). The percentage of positively stained area was analysed in kidney cortex in blinded fashion.

### Antibodies and Immunofluorescence Stainings

Kidney tissues were fixed in 4% formalin for 2 hours at RT and frozen in OCT after dehydration in 30% sucrose overnight. Using 5-10 μm cryosections, slides were blocked in 5% donkey serum followed by 1-hour incubation of the primary antibody, washing 3 times for 5 minutes in PBS and subsequent incubation of the secondary antibodies for 45 minutes. Following DAPI (4′,6′-′diamidino-2-phenylindole) staining (Roche, 1:10.000) the slides were mounted with ProLong Gold (Invitrogen, #P10144). The following antibodies were used: anti-mouse PDGFRa (AF1062, 1:100, R&D), anti-CD10 human (clone HI10a, 1:100, biolegend), anti-HNF4a (clone C11F12, 1:100, Cell Signalling), Pan-Cytokeratin TypeI/II (Invitrogen, Ref. MA1-82041), Dach1 (Sigma, HPA012672), Col1a1 (Abcam, ab34710), ERG (abcam, ab92513), AF488 donkey anti goat (1:100, Jackson Immuno Research), AF647 donkey anti-rabbit (1:200, Jackson Immuno Research).

### Confocal Imaging

Images were acquired using a Nikon AIR confocal microscope using 40× and 60× objectives (Nikon). Raw imaging data was processed using Nikon Software or ImageJ.

### Human Kidney Tissue Microarray

Paraffin-embedded, formalin-fixed kidney specimens from 98 non-tumorous human kidney samples of the Eschweiler/Aachen biobank were selected based on a previously performed PAS staining. Areas were randomly selected per sample and one 2-mm core was taken from each kidney sample using the TMArrayer™ (Pathology Devices, Beecher Instruments, Westminster, USA). Each core was arrayed into recipient block in a 2 mm-spaced grid covering approximately 2.5 square cm, and 5-micron thick sections were cut and processed using standard histological techniques.

### RNA In-Situ Hybridization

In situ hybridization was performed using formalin-fixed paraffin embedded tissue samples and the RNAScope Multiplex Detection KIT V2 (RNAScope, #323100) following the manufacturer's protocol with minor modifications. The antigen retrieval was performed for 22 min at 96° C. instead of 15 min at 99° C. in a water bath. 3-5 drops of pretreatment 1 solution were incubated at RT for 10 minutes after performing antigen retrieval. The washing steps were performed 5 minutes three times. The following probes were used for the RNAscope assay: Hs-PDGFRB #548991-C1, Hs-PDGFRa #604481-C3, Hs-Col1a1 #401891, Hs-COLIA1 #401891-C2, Hs-MEG3 #400821, Hs-NKD2 #581951-C2 (targeting 236-1694 of NM_033120.3), Hs-Postn #409181-C2 and 409181-C3, Hs-Pecam1 #487381-C2, Hs-Cc119 #474361-C3, Hs-Cc121 #474371-C2, Hs-Notch3 #558991-C2, Mm-Col1a1 #319371, Mm-PDGFRa #480661-C2, Mm-PDGFRb #411381-C3.

Image Quantification—ISH Image Analysis

Systematic random sampling was applied to subsample at least 3 representative tubulo-interstitial areas per image. Next, every fluorescent dot (transcript) was manually annotated using the cell counting tool from Fiji (Max Planck Institute of Molecular Cell Biology and Genetics, Dresden, Germany). Single nuclei were then isolated using an in-house made tool (https://gitlab.com/mklaus/segment_cells_register_marker) based on watershed (limits: 0.1-0.4) to identify neighbouring nuclei, edge detection for incomplete objects and object size selection (limits: 12-180 μm2). The total number of individual dots was then retried for every isolated nucleus. Dots located outside of nuclei were not included in this analysis, as the complexity of kidney morphology combined with high cellular density prevented us to determine the origin of non-nuclear transcripts. For Meg3 and NKD2 analysis of PDGFRa/b cells images were analyzed using QPath after segmenting the nuclei and counting cells based on >1 pos. spot per imaging channel. For Col1a1-IF quantification or NKD2-ISH quantification images were split in RGB channels and the integrated fluorescent density was determined per image using ImageJ.

### Quantitative RT-PCR

Cell pellets were harvested and washed with PBS followed by RNA extraction according to the manufacturer's instructions using the RNeasy Mini Kit (qiagen). 200 ng total RNA was reverse transcribed with High-Capacity cDNA Reverse Transcription Kit (Applied Biosystems). qRT-PCR was carried out with iTaq Universal SYBR Green Supermix (Biorad) and the Bio-Rad CFX96 Real Time System with the C1000 Touch Thermal Cycler. Cycling conditions were 95° C. for 3 minutes, then 40 cycles of 95° C. for 15 seconds and 60° C. for 1 minute, followed by 1 cycle of 95° C. for 10 seconds. GAPDH was used as a housekeeping gene. Data were analyzed using the 2-CT method. The primers used are listed in Table 2.

TABLE 2
List of RT-PCR primer sequences (human)

| Genes | Forward primer | Reverse primer |
|---|---|---|
| collagen type | 5′-CCCAGCCACAAAGAGICTACA | 5′-ATTGGTGGGATGTCTTCGTCT |
| I alpha 1 | | |
| fibronectin 1 | 5′-ACAAACACTAATGTTAATTGC | 5′-TCGGGAATCTTCTCTGTCAGC |
| | CCA | |
| actin alpha 2, | 5′-ACTGOCTTGGTGTGTGACAA | 5′-CACCATCACCCCCTGATGTC |
| smooth muscle | | |
| posm | 5′-GGCTCATAGTCGTATCAGGGG | 5′-GGTGCCCAAAATCTGTTGAAG |
| | | G |
| Nkd2 | 5′CACGTCAGGAGOCCCAGTA | 5′-TGTAGTTCTCAATCCCGGCG |
| cFos | 5′-GGAGAATCCGAAGGGAAAGGA | 5′-AGTTGGTCTGTCTCCGCTTG |
| Ogn | 5′-CCATAATGCCCTGGAATCCGT | 5′-CAATGCGGTCCCGGATGTAA |
| GAPDH | 5′-GAAGGTGAAGGTCGGAGICA | 5′-TGGACTCCACGACGTACTCA |

Generation of a Human PDGFRb+ Cell-Line PDGFRb+ cells were isolated from healthy human kidney cortex of a nephrectomy specimen (71 years old male patient) by generating a single cell suspension (as above) followed by MACS separation (Miltenyi biotec, autoMACS Pro Separator, #130-092-545, autoMACS Columns #130-021-101. For the isolation the single cell suspension was stained in two steps using first a specific PDGFRb antibody (R&D #MAB1263 antibody, dilution 1:100) followed by a second incubation step with an Anti-Mouse IgG1-MicroBeads solution (Miltenyi, #130-047-102). Following MACS cells were cultured in DMEM media (Thermo Fisher #31885) added 10% FCS and 1% penicillin/Streptomycin for 14 days and immortalized using SV40LT and HTERT as follows. Retroviral particles were produced by transient transfection of HEK293T cells using TransIT-LT (Mirus). Two types of amphotropic particles were generated by co transfection of plasmids pBABE-puro-SV40-LT (Addgene #13970) or xlox-dNGFR-TERT (Addgene #69805) in combination with a packaging plasmid pUMVC (Addgene #8449) and a pseudotyping plasmid pMD2.G (Addgene #12259). Retroviral particles were 100× concentrated using Retro-X concentrator (Clontech) 48 hrs post-transfection. Cell transduction was performed by incubating the target cells with serial dilutions of the retroviral supernatants (1:1 mix of concentrated particles containing SV40-LT or rather hTERT) for 48 hrs. Subsequently the infected PDGFRb+ cells were selected with 2 μg/ml puromycin at 72 h after transfection for 7 d.
Culturing Human Induced Pluripotent Stem Cell (iPSC) Derived Kidney Organoids

Human iPSC-15 clone 0001 was received from the Stem Cell Facility of the Radboud University Center, Nijmegen, The Netherlands. Human iPSC were grown on Geltrex-coated plates using E8 medium (Life Technologies). Upon 70-80% confluency, iPSC were detached using 0.5 mM EDTA and cell aggregates were reseeded by splitting 1:3. Human iPSC were differentiated using a modified protocol based on Takasato et al. (Nature, 2015) and seeded at a density of 18,000 cells per cm2 on geltrex-coated plates (Greiner). Differentiation towards intermediate mesoderm was initiated using CHIR99021 (6 μM, Tocris) in E6 medium (Life Technologies) for 3 and 5 days, followed by FGF 9 (200 ng/ml, RD systems) and heparin (1 μg/ml, Sigma Aldrich) supplementation in E6 up to day 7. After 7 days of differentiation, cell aggregates (300,000 cells per organoid, mixture of 3 and 5 day CHIR-differentiated cells) were cultured on Costar Transwell inserts to stimulate self-organizing nephrogenesis using E6 differentiation medium. On day 7+18 the kidney organoids were used for siRNA knockdown experiments as described below.

siRNA Knockdown of NKD2 in Human iPSC-Derived Kidney Organoids

NKD2 siRNA knockdown was carried out according to the manufacturer protocol (DharmaFECT transfection reagent and NKD2-specific smartpool siRNA, both Horizon Discovery). The transfection master mix and scrambled controls were prepared in Essential 6 medium (Gibco) and added to the organoids. After an initial incubation of 24 h, the transfection master mixes were refreshed and IL-1β (Sigma-Aldrich) was added at a concentration of 100 ng/ml to induce fibrosis. The IL-1β exposure together with refreshing the transfection master mix was repeated every 24 h for two upcoming days. 96 h post transfection initiation, the organoids were harvested and processed for paraffine sectioning. Fluorescence in-situ hybridisation (FISH) and immunofluorescence staining was performed as described above.

### TGFb-Treatment Experiments

TGFb (100-21-10UG, Peprotech) at a concentration of 10 ng/ml in PBS was added to 75% confluent PDGFRb cells for 24 hours after 24 hours serum starvation with 0.5% FCS containing medium. For inhibitor experiments with T-5224 the inhibitor (or vehicle) was added to the culture wells 1 hour before adding TGFb. All experiments were performed in triplicates.

### AP-1 Inhibitor Treatment

T-5224 (c-Fos/AP-1inhibitor, Cayman Chemicals, #22904) was dissolved in DMSO and stored at −80° C. DMSO was always added in the same proportions to control wells.

### Cell Proliferation (WST-1 Assay)

WST-1 assay with PDGFRb-cells was performed in 96-wells as recommended by the manufacturer (Roche Applied Science). In brief, 1×10\hat{ }4 PDGFRb cells were seeded into each well of 96-well plates and the cells were treated with T-5224 or vehicle (DMSO) with the indicated concentrations in triplicates. Cells were incubated with WST-1 reagent for 2 h before harvesting at the indicated time points. Both 450 nm and 650 nm (as a reference) absorbance were measured.

sgRNA: CRISPR-Cas9 Vector Construction, Virus Production and Transduction

The NKD2-specific guide RNA (forward 5′-CACCGACTCCAGTGCGATGTCTCGG-3′; reverse 5′-AAACCCGAGACATCGCACTGGAGTC-3′) were cloned into pL-CRISPR.EFS.GFP (Addgene #57818) using BsmBI restriction digestion. Lentiviral particles were produced by transient co transfection of HEK293T cells with lentiviral transfer plasmid, packaging plasmid psPAX2 (Addgene #12260) and VSVG packaging plasmid pMD2.G (Addgene #12259) using TransIT-LT (Mirus). Viral supernatants were collected 48-72 hours after transfection, clarified by centrifugation, supplemented with 10% FCS and Polybrene (Sigma-Aldrich, final concentration of 8 μg/ml) and 0.45 μm filtered (Millipore; SLHP033RS). Cell transduction was performed by incubating the PDGFRB cells with viral supernatants for 48 hrs. eGFP expressing cells were single cell sorted into 96 well plates. Colonies expanded were assessed for mutations with mismatch detection assay: gDNA spanning the CRISPR target site was PCR amplified and analyzed by T7EI digest (T7 Endocnuclease, NEB M0302S). To determine specific mutation events on both alleles within the clones grown, the PCR product was subcloned into the pCR™ 4Blunt-TOPO vector (Thermo Scientific K287520). Minimum 6 colonies per CRISPR-clone were grown and sent for sanger sequencing (Clone C2: 30 colonies have been sequenced). Western blot was performed to demonstrate complete knockout of NKD2.

### Western Blot

Western blots were performed according to standard protocols. In brief, cell lysates were prepared by RIPA buffer with protease inhibitor cocktail (Roche). The protein concentrations of the lysates were quantified using BCA assay (#23225, Pierce, ThermoScientific). The protein lysates were heated for 5 min at 95° C. in 4× SDS sample loading buffer (BioRad) and loaded into 10% SDS-Page gels. Afterwards samples were transferred onto PVDF membranes and the blots were probed with primary antibody in 5% Blotto (Thermo Fisher): (1:3000 rabbit anti-human NKD2 polyclonal antibody, Invitrogen PA5-61979) for 2 hours, followed by incubation with secondary antibody for 1 hour after washing (1:5000 horseradish-peroxidase-HRP-conjugated anti rabbit, Vector Laboratories) and developed using Pierce™ ECL Western Blotting Substrate A and B. Mouse monoclonal anti-GAPDH antibody (NovusBiologicals NB300-320; 1:1000) followed by HRP conjugated anti mouse secondary antibody (Vector laboratories) was used as a loading control.

### Lentiviral Overexpression of Nkd2

NKD2 vector construction and generation of stable NKD2-overexpressing cell lines. The human cDNA of NKD2 was PCR amplified using the primer sequences 5′-atggggaaactgcagtcgaag-3′ and 5′ ctaggacgggtggaagtggt-3′. Restriction sites and N-terminal 1×HA-Tag have been introduced into the PCR product using the primer 5′-cactcgaggccaccatgtacccatacgatgttccagattacgctgggaaactgcagtcgaag-3′ and 5′-acggaattcctaggacgggtggaagtg-3′. Subsequently, the PCR product was digested with XhoI and EcoRI and cloned into pMIG (pMIG was a gift from William Hahn (Addgene plasmid #9044; http://n2t.net/addgene: 9044; RRID: Addgene_9044). Retroviral particles were produced by transient transfection in combination with packaging plasmid pUMVC (pUMVC was a gift from Bob Weinberg (Addgene plasmid #8449)) and pseudotyping plasmid pMD2.G (pMD2.G was a gift from Didier Trono (Addgene plasmid #12259; http://n2t.net/addgene: 12259; RRID: Addgene_12259)) using TransIT-LT (Mirus). Viral supernatants were collected 48-72 hours after transfection, clarified by centrifugation, supplemented with 10% FCS and Polybrene (Sigma-Aldrich, final concentration of 8 μg/ml) and 0.45 μm filtered (Millipore; SLHP033RS). Cell transduction was performed by incubating the PDGFβ cells with viral supernatants for 48 hrs. eGFP expressing cells were single cell sorted.

### Bulk RNA Sequencing

RNA was extracted according to the manufacturer's instructions using the RNeasy Mini Kit (QIAGEN). For rRNA-depleted RNA-seq using 1 and 10 ng of diluted total RNA, sequencing libraries were prepared with KAPA RNA HyperPrep Kit with RiboErase (Kapa Biosystems) according to the manufacturer's protocol. Sequencing libraries were quantified using quantitative PCR (New England Biolabs, Ipswich, USA), equimolar pooled, final pool is normalized to 1.4 nM and denatured using 0.2 N NaOH and neutralized with 400 nM Tris pH 8.0 prior to sequencing. Final sequencing was performed on a NextSeq platform (Illumina) according to the manufacturer's protocols (Illumina, CA, USA).

ATAC-Seq Preparation 5000-7500 PDGFRa/b pos cells were FACS sorted from freshly isolated UUO kidneys as described above, washed twice with cold PBS and centrifuged at 500 g for 5 minutes. Cell pellets were then lysed in 50 μl ice-cold lysis buffer (10 mM Tris-HCl, pH7.5; 10 mM NaCl, 3 mM MgCl2, 0.08% NP40 substitute [74385, Sigma], 0.01% Digitonin [G9441, Promega]), and immediately centrifuged at 500 g for 9 minutes. Pellets were resuspended in 50 μl of a transposase reaction mix, including 25 μl 2×TD buffer (20 mM Tris-HCl, pH7.6, 10 mM MgCl2, 20% DMF), 0.5 μl tagment DNA enzyme 1 [15027865, Illumina] and 24.5 μl nuclease free water. The transposition reaction was incubated at 37° C. for 30 min at 350 rpm in a thermoshaker. Following this, the transposed DNA was purified using a MinElute Reaction Cleanup kit (28204, Qiagen) and eluted in 15 μl nuclease free water. Transposed DNA was amplified by PCR (14 cycles total) using NEB Next 2× Master mix (M0541S; New England Biolabs) with custom Nextera PCR primers. The first PCR was performed with 50 μl volume and 6 cycles using NEB Next 2× Master mix and 1.25 μM custom primers; the second RT-PCR was performed with 15 μl volume for 20 cycles using 5 μl (10%) of the pre-amplified mixture plus 0.125 μM primers to determine the number of additional cycles needed as described previously. The amplified DNA library was purified using MinElute PCR Purification kit (28004, Qiagen) and eluted in 20 μl of 10 mM Tris-HCl (pH 8). The quality of the library was visualized by Agilent D1000 ScreenTape on 2200 TapeStation system (Agilent Technologies). The ATAC-seq libraries were loaded on Illumina NextSeq 500 for 75-bp paired-end sequencing.

### Smart-Seq2 Data Processing

The initial single-cell transcriptomic data was processed at the Eukaryotic Single-Cell Genomics Facility at the Science for Life Laboratory in Stockholm, Sweden. Obtained reads were mapped to the mm10 build of the mouse genome (concatenated with transcripts for eGFP and the ERCC spike-in set) to yield a count for each endogenous gene, spike-in, and eGFP transcript per cell. Ribosomal RNA genes, ribosomal proteins and ribosomal pseudo-genes were filtered out. We noticed that cells that did not feature any alignments assigned to either eGFP or PDGFRb clustered into a single cluster after unsupervised cell clustering (see below).

Therefore, we opted to remove those cells, and performed all analysis and clustering without considering those cells (17 cells).

### 10× Single Cell RNA-Seg Data Processing

Fastq files were processed using Alevin and Salmon (Alevin parameters-1 ISR, Salmon version 0.13.1), using Gencode v29 human transcriptome and Gencode vM20 mouse transcriptome as reference transcriptomes. Alevin's expected Cells parameter was set according to thrice the number of cells estimated according to the knee-method applied to the read counts per cell barcodes distribution. Therefore, UMI count matrix produced by Alevin produced a large number of putative cells which we could filter later (see next paragraph).

10× scRNA-Seg Cell Filtering

We moved ribosomal RNA genes (0-1% on average of detected RNA content per cell) and mitochondrially-encoded genes (0-80% on average of detected RNA content per cell) from the main gene expression matrix. Mitochondrially-encoded genes were removed to avoid introducing unwanted variation between cells that might be solely dependent on changes in mitochondrial content. log 10 (total UMI counts per cell) distribution from the count matrix produced by Alevin (see above) typically showed a bimodal distribution, therefore log 10 (total UMI counts per cell) were clustered into two clusters using mclust R package v5.4.3 setting modelNames to “E”. Cells that belong to the cluster with the higher counts were kept. Then cells were filtered based on mitochondrial RNA content and bias toward highly expressed genes as follows: (1) cells were clustered into two clusters using a bivariate Gaussian mixture with two components learned on log 10 (total UMI counts per cell) and percent of mitochondrial UMI per cell. Clustering was performed using the R package Mclust setting modelNames to “EII”. Cells falling into the cluster with higher mitochondrial content cells were excluded. This filtering step was followed only for libraries which showed a clear bimodal distribution of mitochondrial content (only three 10× libraries in this study) (2) The total number of UMIs per cell should correlate with the total number of unique detected genes. Cells that do not follow this relationship (outliers) were filtered by clustering nuclei using a bivariate Gaussian mixture model on log 10 (total UMI counts) and log 10 total unique detected genes using the mclust R package setting modelNames to “VEV”, “VEE”. (3) Cells whose percent of total counts in the top 500 genes represented more than 5 times absolute median deviation for all cells were removed. (4) Finally, to exclude cells comprised mainly of ribosomal proteins and pseudo-genes, we removed cells whose percent of ribosomal protein and pseudo-gene expression represented more than 5 absolute median deviations of all other cells. Mitochondrial-based filtering was not performed for CD10+ libraries since libraries from proximal tubule epithelial cells are expected to result in a high number of mitochondrial reads. Note that not all filtering steps were performed for all libraries as this depends on each library's quality and UMI-cell-gene distribution.

### Human 10× Single Cell Data Integration Strategy

Upon initial analysis of our data, we noted several points: (1) Cell types are not guaranteed to be equally represented across patients and across conditions (healthy or CKD). This is because the cell types captured in any single 10× Chromium run are determined by random sampling of cells. (2) Both healthy and CKD patient samples consist of cells in healthy and disease states, since this categorization is based on clinical parameters and not on molecular data or a controlled in vitro experiment. We would expect mainly a change in proportion of healthy and disease cell states between healthy and diseased patient samples. (3) Samples from different patients were processed and prepared on different days as dictated by the surgery schedule at the Eschweiler hospital. Therefore, potential technical (batch) effects could not be controlled on the experimental side. (4) The ability to discover highly resolved cell clusters in under-represented cell types might be affected by class imbalance since certain cell types may be significantly more abundant than others, and the size of the dataset (number of cells) which affects clustering results using unsupervised modularity-based graph clustering algorithms.

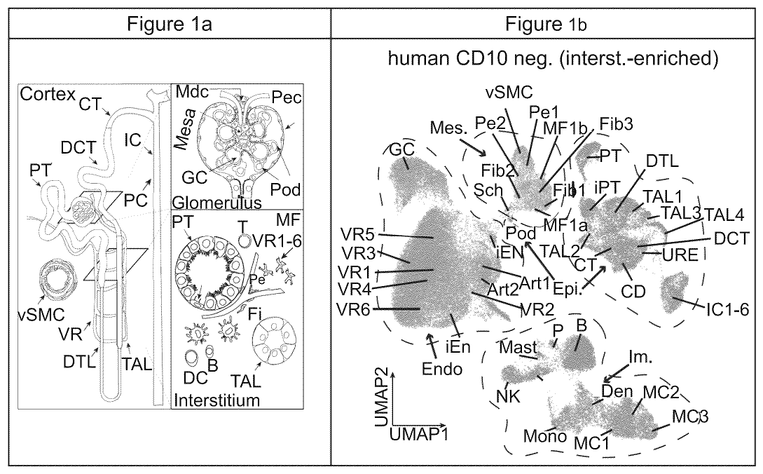

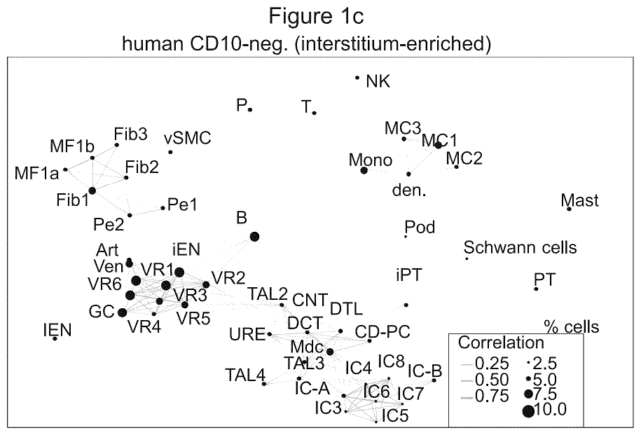

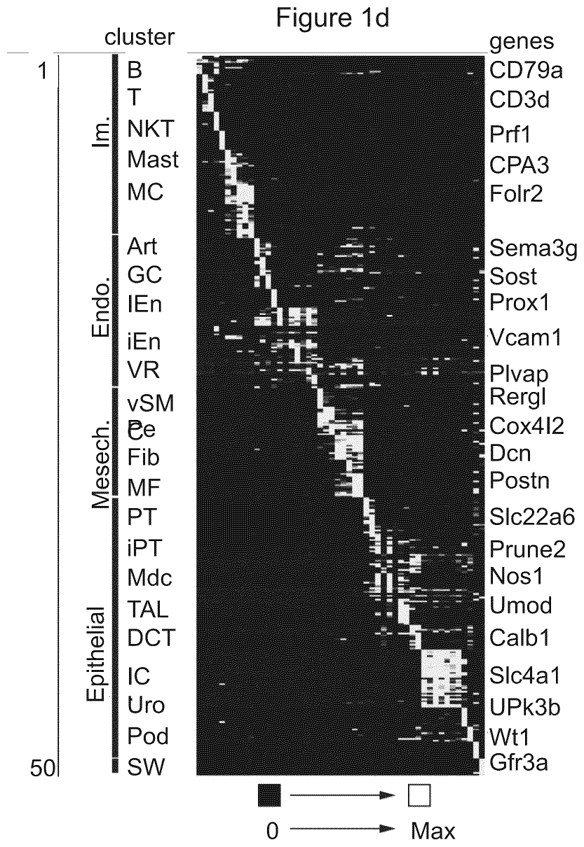

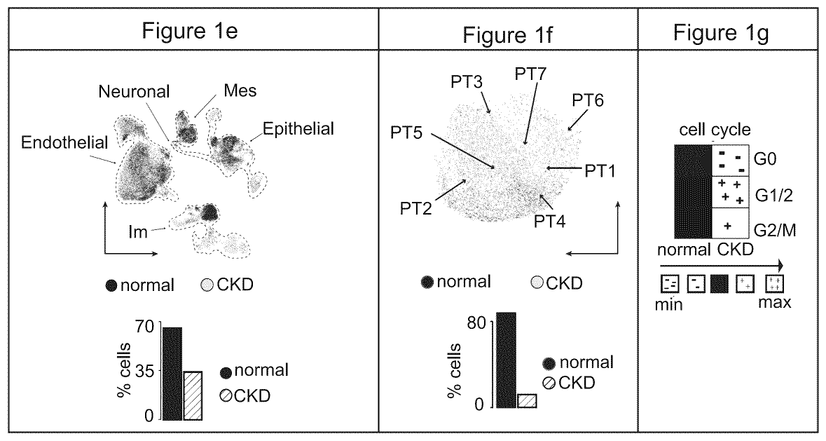

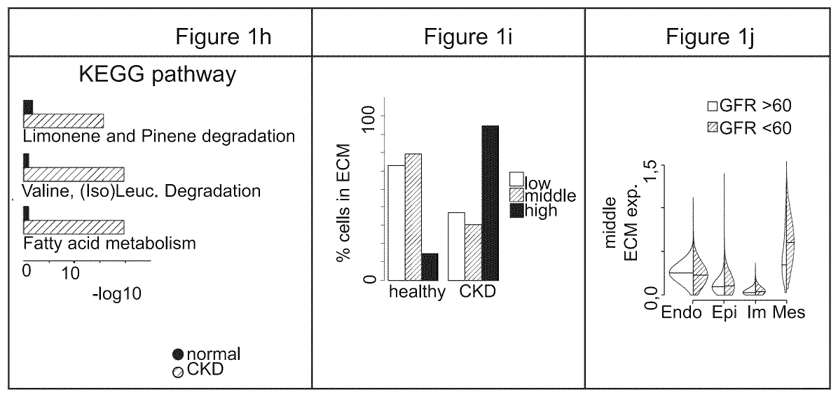

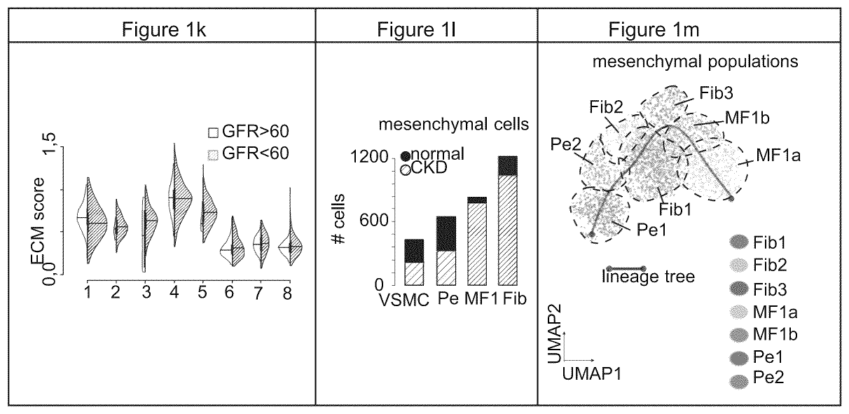

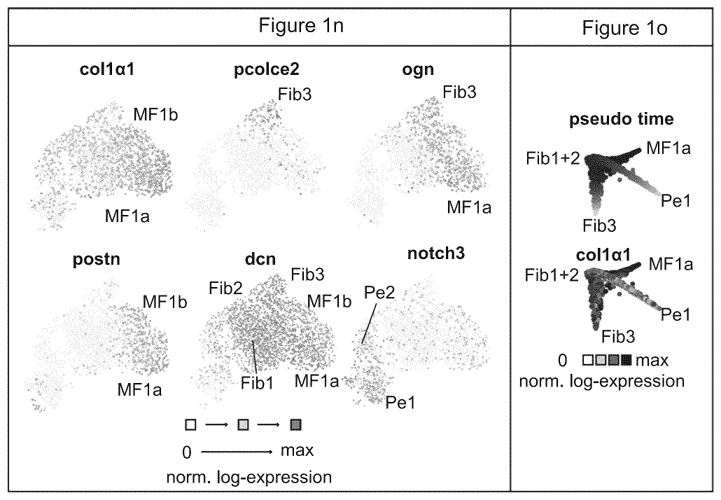

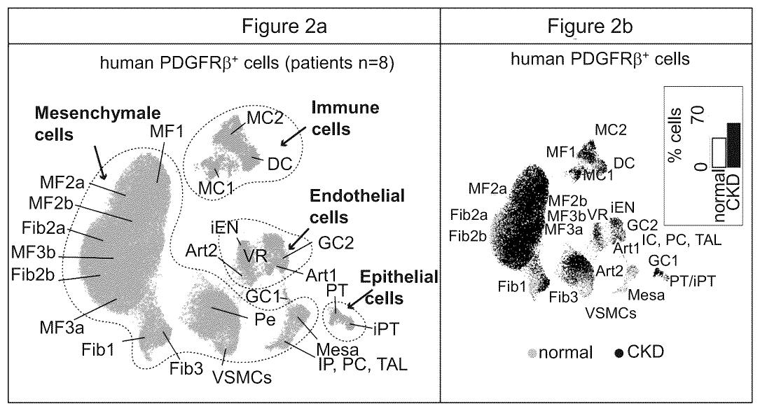

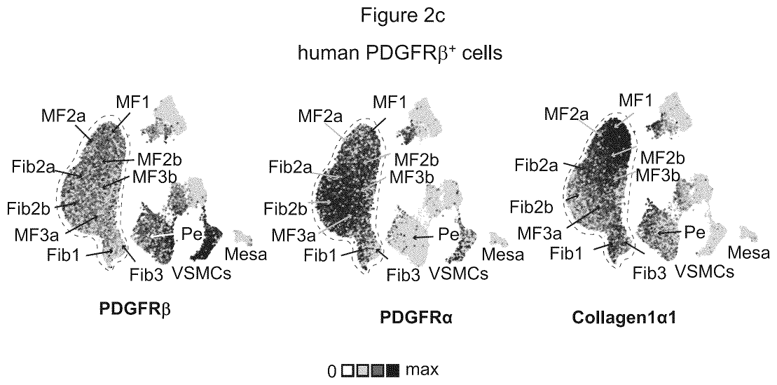

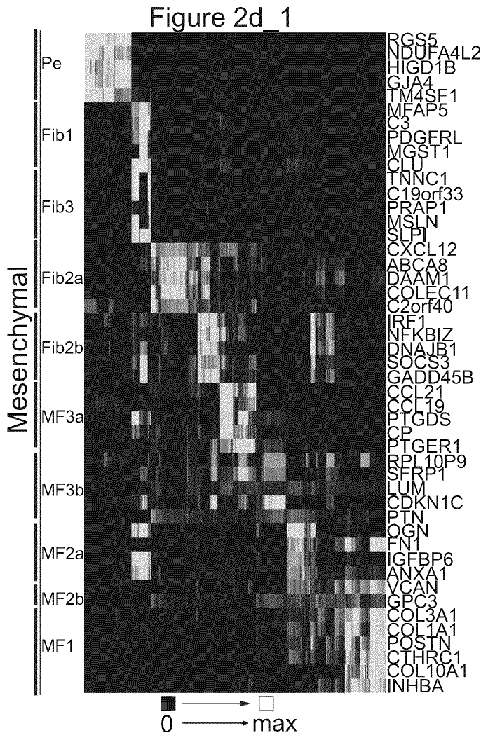

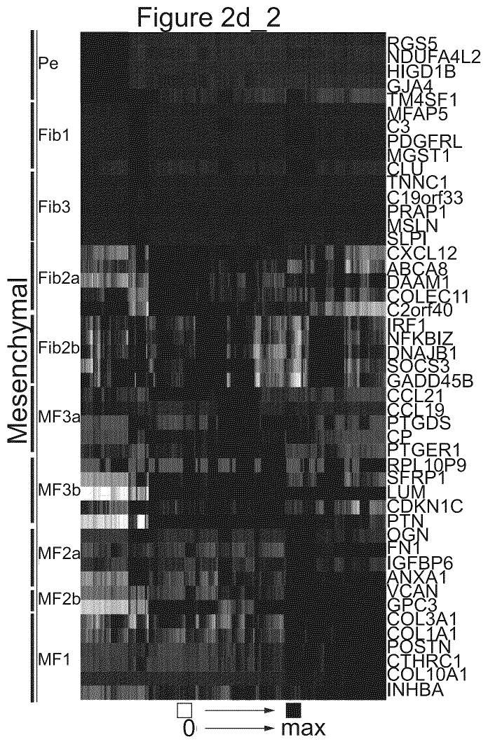

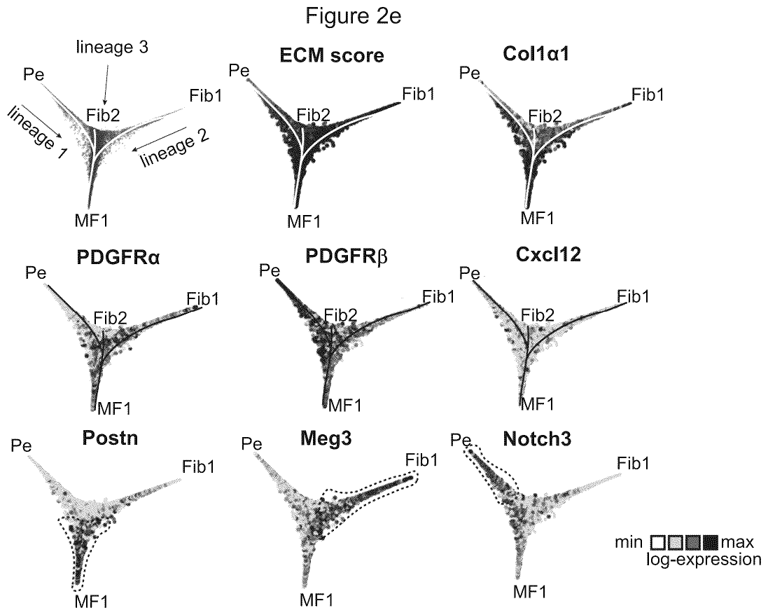

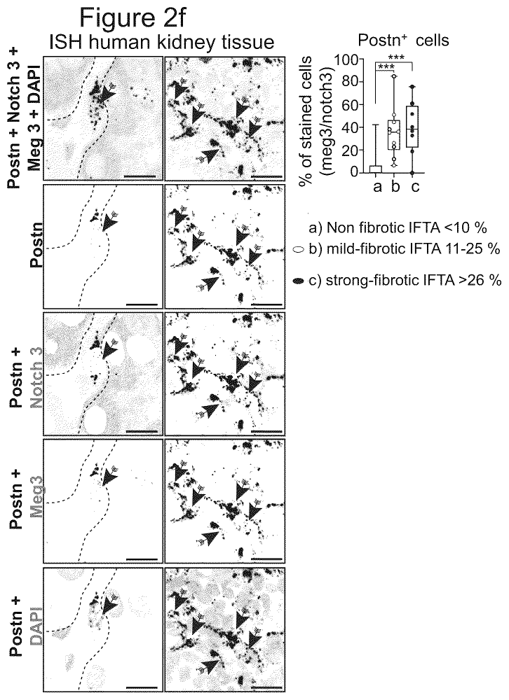

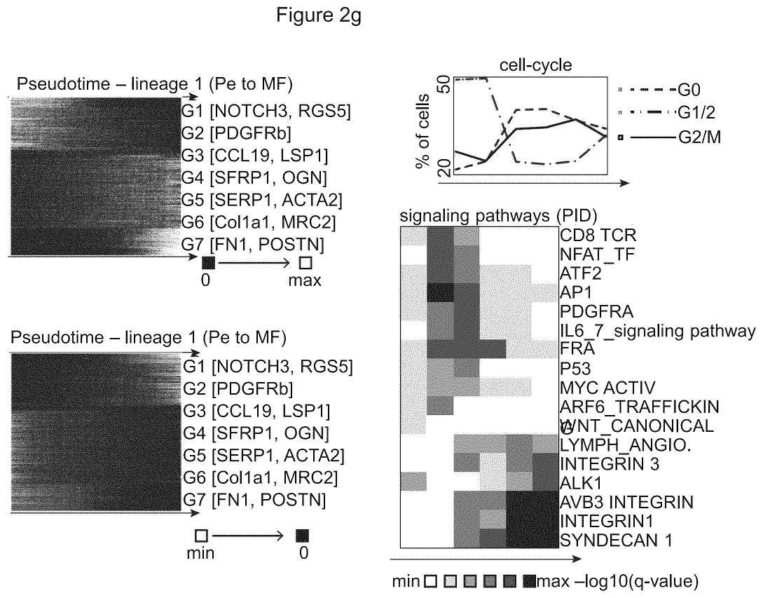

The experimental strategy involved obtaining separate libraries from CD10+ and CD10− cell fractions, which was designed to mitigate class imbalance on the level of cell type capturing frequency by the 10× Chromium protocol. To further mitigate the points discussed above we aimed to (1) cluster the data on a local level while keeping global information on the relation between cell types intact and (2) to correct for potential technical (batch) differences between samples while retaining important differences, such as different cell types or different states of cell types due to disease. To do so, we followed a strategy comprised of the following steps:
Step One: After quality control and cell filtering (see above), cells in each 10× library were clustered separately and each cell cluster was assigned to one of 6 major cell types: CD10+ epithelial, CD10-epithelial, Immune, Endothelial, Mesenchymal and Neuronal cells.
Step Two: For each one of the 6 major cell types, cells from all 10× libraries were integrated together. Variability between cells due to technical reasons was corrected and cells were clustered using unsupervised graph clustering. This process resulted in 6 separate endothelial, CD10+ epithelial, CD10− epithelial, mesenchymal, immune and neuronal maps. Each map composed of cells from multiple 10× libraries.
Step Three: We integrated 3 single cell maps for: (1) CD10+ cells (proximal tubule/FIG. 1), (2) CD10− cells (proximal tubule-depleted/FIG. 1) and (3) PDGFRb+ cells (mesenchymal/FIG. 2), by combining single cell expression (UMI counts) and clustering information from all main cell type individual maps of each data set from Step Two. All plots in the manuscript are thereafter reproducible from those 3 integrated maps.

This approach accomplished local clustering and technical variability removal, and allowed for high resolution discovery of cell states regardless of highly variable cell cluster sizes. The smallest cluster consisted of 24 cells, while the largest cluster consisted of 5355 cells. Relative to “a high-level clustering followed by sub-clustering” approach, our approach produces highly resolved clusters in a data-driven unbiased manner, while avoiding the question of which clusters to subcluster altogether. We note that Zeisel et al. followed a somewhat similar data integration approach.

Overall, this approach was biologically informed, and allowed us to correct for potential technical effects during cell clustering such that almost all cell clusters contained cells from more than one patient/library, while preserving interesting differences between patients such as diseased cell states (for example injured Proximal tubule cells), differences in (myo)fibroblast states and differences in ECM expression.

### Mouse 10× Single Cell Data Integration Strategy

Mouse 10× data were analyzed and integrated in the same way as described for human data. The script used to produce the integrated map is available here: https://raw.githubusercontent.com/mahmoudibrahim/KidneyMap/master/make_intergrated maps/mouse_PDGFRABpositive.r

### Mouse Smart-Seg2 Single Cell Data Integration Strategy

Since single cell plate sorting was performed such that cells from all three timepoints were equally represented in all plates, no further batch effect mitigation was performed during the analysis. Variable genes were determined using the Scran R package decompose Var function, after running the trendVar function on the ERCC transcripts6. Genes with an FDR value <0.01 and biological variance component >1 were kept as highly variable genes. Using those variable genes we followed the same clustering approach as described for the 10× Chromium data, but we ran only 2 clustering iteration and did not vary the number of nearest neighbours. Script used for analysis of mouse Smart-Seq2 data is available here: https://github.com/mahmoudibrahim/KidneyMap/blob/master/make_intergrated_maps/mouse_PDGFRBpositive.r.

### Cluster Annotation

A gene ranking per cluster was produced using the sortGenes function in the genesorteR R package setting binarizeMethod to “adaptiveMedian” (Smart-Seq2 Data) or to “naive” (10× Data). We then annotated our highly resolved cell clusters manually based on prior knowledge and information from literature. There were 50 such clusters in CD10-data, 7 clusters in CD10+ data, 26 clusters in PDGFRb+ human data, 10 clusters in mouse Smart-Seq2 data and 10 clusters in mouse PDGFRa+/b+ data. At that highly-resolved level (level 3), a cell cluster can either represent a bona fide cell type or a different cell state. Thus, we also grouped those highly-resolved cell clusters into canonical cell types based on our annotation. This resulted in 29 cell types in CD10− map, 1 cell type in CD10+ map, 16 cell types in PDGFRb+ map, 5 cell types in mouse PDGFRa+/b+ map and 6 cell types in Smart-Seq2 mouse PDGFRb+ map. We then further annotated the cell clusters as either epithelial, endothelial, mesenchymal, immune or neuronal for plot and figure annotation in order to enable easier data interpretation.

### UMAPs and Diffusion Maps

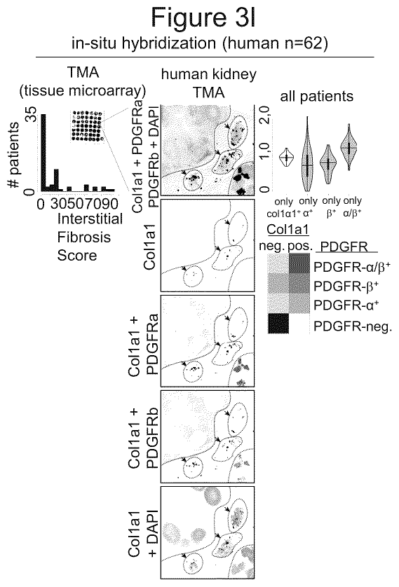

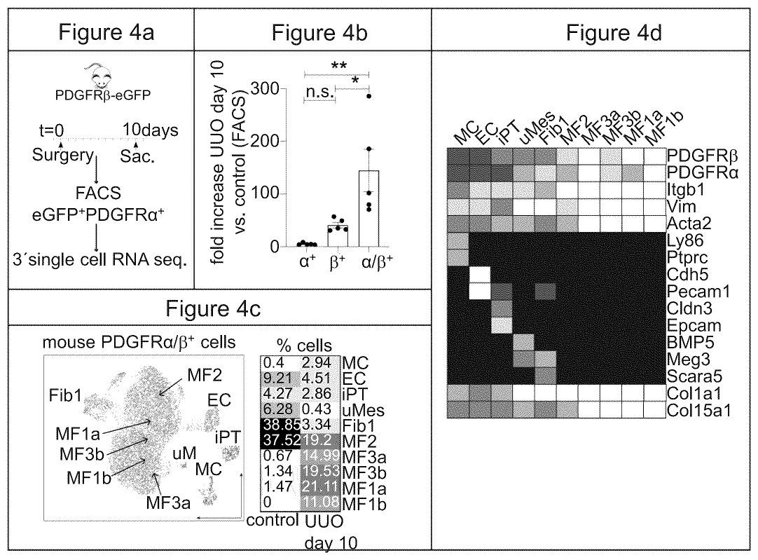

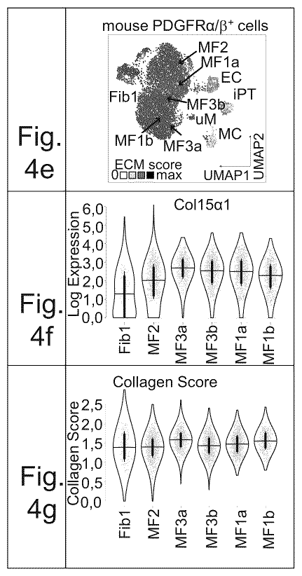

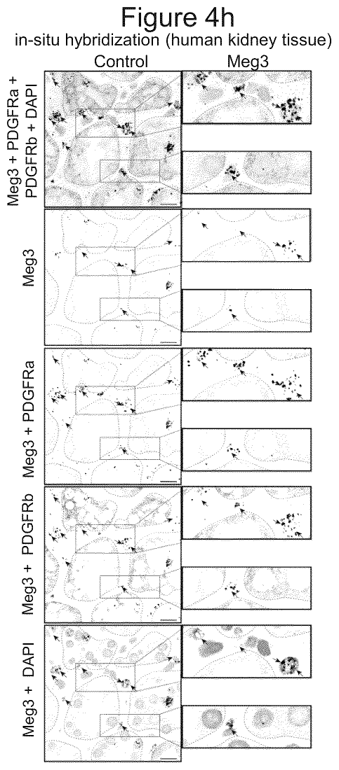

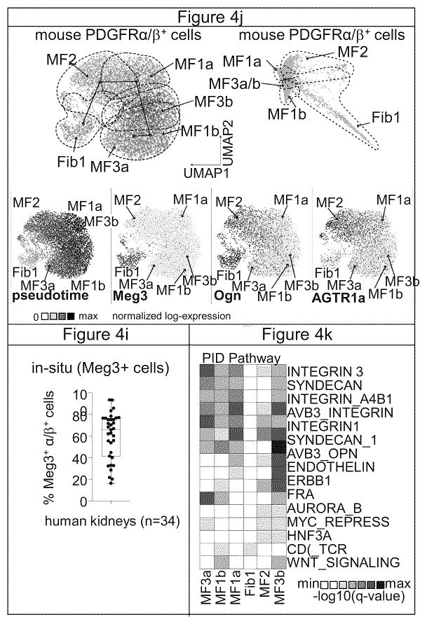

Integrated full-map UMAP projections (FIG. 1, 2, 3, 4, 5) were generated via the UMAP Python package (https://github.com/lmcinnes/umap) on the reduced corrected dimensions returned from fastMNN setting min_dist to 0.6 and the number of neighbours to square root the number of cells. Local UMAP projections (FIG. 1, FIG. 4) were produced setting min_dist to 1, as those parameters tend to produce more geometrically accurate embeddings (see https://umap-learn.readthedocs.io/en/latest/). Diffusion Maps were produced using the Destiny R package (https://github.com/theislab/destiny) also using the reduced dimensions returned from fastMNN as input and setting the number of neighbours to square root the number of cells. We tested various randomization seeds for UMAP and Diffusion Map and various Diffusion Map distance metrics (as recommended in the Destiny R package manual) and confirmed that no qualitative difference occurs in the resulting single cell projections.

### Lineage Trees/Trajectories and Pseudotime

The Slingshot R package was used for lineage tree inference and pseudotime cell ordering inference based on the UMAP/Diffusion Map projection. The cell clustering (Step Two from integration strategy, see above) was used as input cell clusters. Start and end clusters were chosen based on reasonable expectation given our prior knowledge as discussed and recommended in Street et al. (for example, myofibroblast is the end cluster in a pericyte/fibroblast/myofibroblast map).

### Gene Dynamics Along Pseudotime

Genes whose expression varied with cell ordering were defined as those whose normalized expression correlated with cell ordering as quantified by the spearman correlation coefficient at a Bonferroni-Hochberg corrected p-value cutoff of 0.001. Gene clusters and expression heatmaps (for example, FIG. 2f-top) were produced by ordering cells along the pseudotime predicted by SlingShot and using the genesorteR function plotMarkerHeat. This function clusters genes using the k-means algorithm, and we set the plot and clustering to average every 10 cells along pseudotime. Pathway enrichment and cell cycle analyses were calculated by grouping every 2000 cells along pseudotime.

### Pathway Enrichment and Gene Ontology Analysis

For the single-cell data, we used KEGG pathway and PID pathway data downloaded in November 2019 from MSigDB 327,28 as “.gmt” files. Pathway enrichment analysis was performed using the clusterProfiler R package using the top 100 genes for each cell cluster/group as defined by the sortGenes function from the genesorteR package. The enricher function was used setting minGSSize to 10 and maxGSize to 200. The top 5 terms by q-value for each cell cluster/group were plotted as heatmaps of −log 10 (q-value). Gene Ontology Biological Process analysis was performed on the top 200 genes via the same method. The enricher function was used setting minGSSize to 100 and maxGSize to 500. To compare pathway activity between NKD2+ and NKD2− mesenchymal cells, we used PROGENy to estimate the activity of 14 pathways in a single-cell basis, using the top 500 most responsive genes from the model as it is recommended from a benchmark study.

### Cell Cycle Analysis

Cell cycle analysis was done following the method used in Macosko et al. and explained in the tutorial by Po-Yuan Tung (https://jdblischak.github.io/singleCellSeq/analysis/cell-cycle.html, date: Jun. 7, 2015), using normalized gene expression as input and setting the gene correlation value to 0.1. We used cell cycle gene sets provided in from Yang et al., To quantify enrichment/depletion of single cell cycle assignments (FIG. 1g), we plot the log 2 fold-change of those frequencies relative to the average frequency obtained by randomizing the true frequency matrix 1000 times while keeping row and column sums constant. Randomization was performed using the R package Vegan (https://CRAN.R-project.org/package=vegan). Positive numbers indicate enrichment relative to what would be expected by chance, negative numbers indicate depletion.

### ECM and Collagen Score

The expression of core matrisome genes provided in Naba et al. were summarized based on normalized gene expression data using the same method used for cell cycle analysis.

### Gene Expression Heatmaps

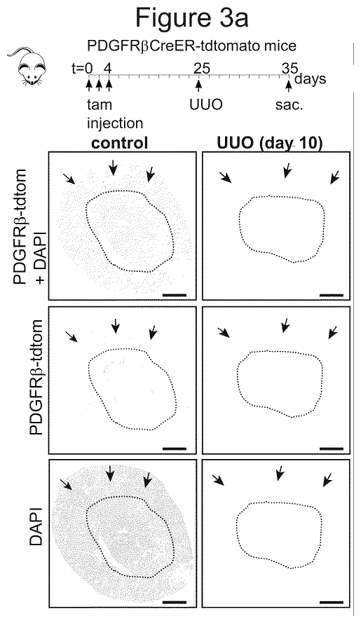

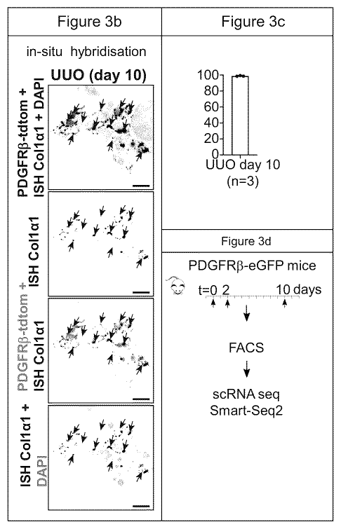

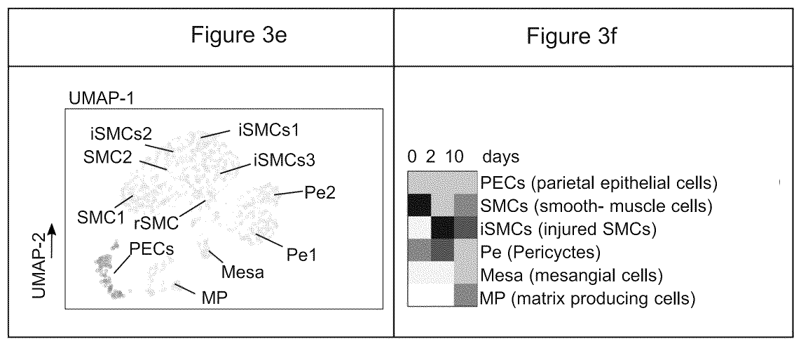

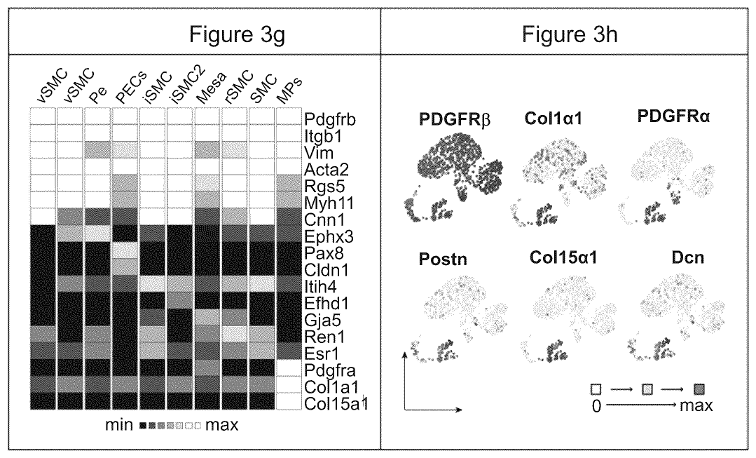

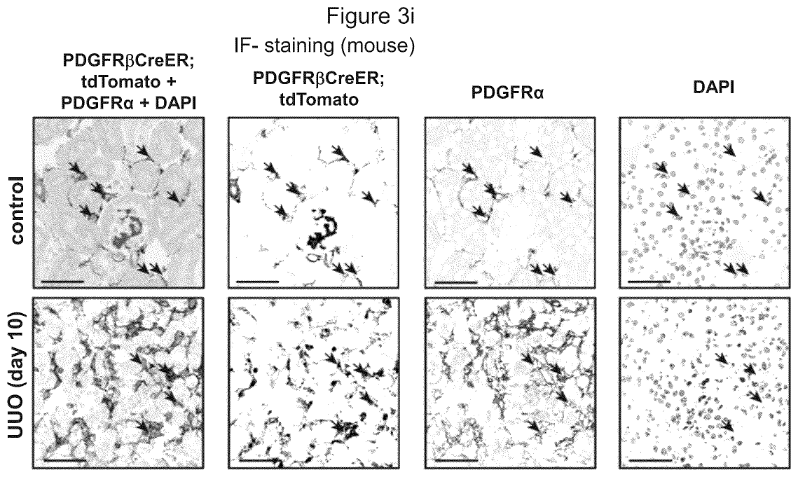

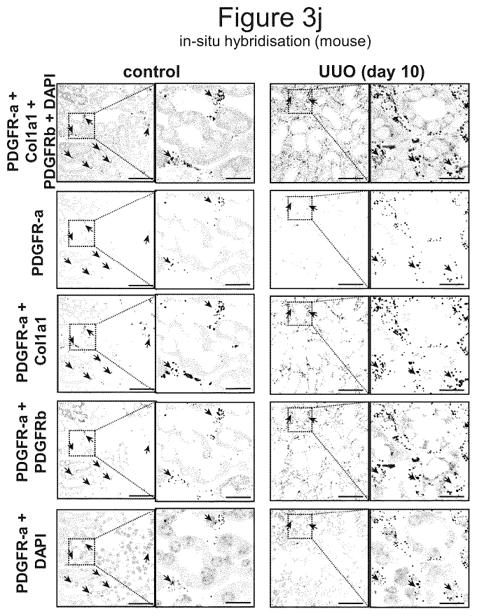

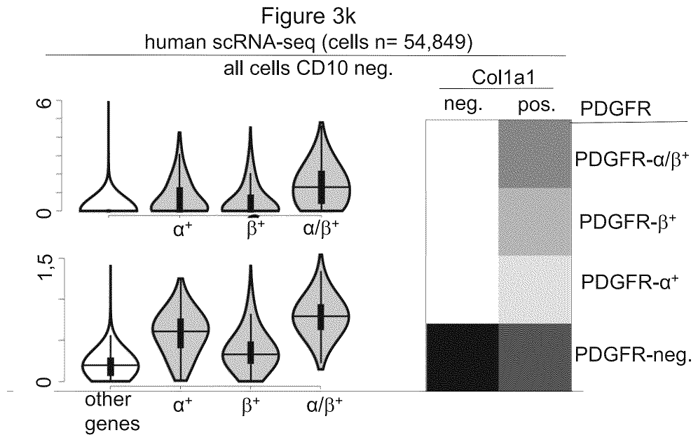

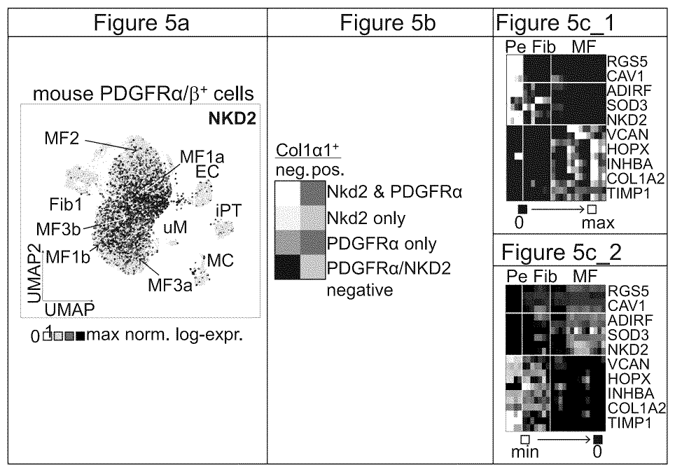

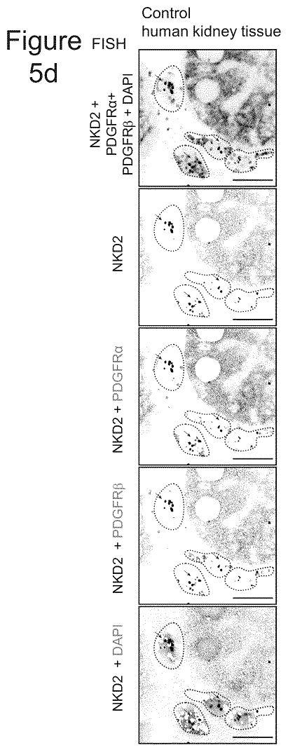

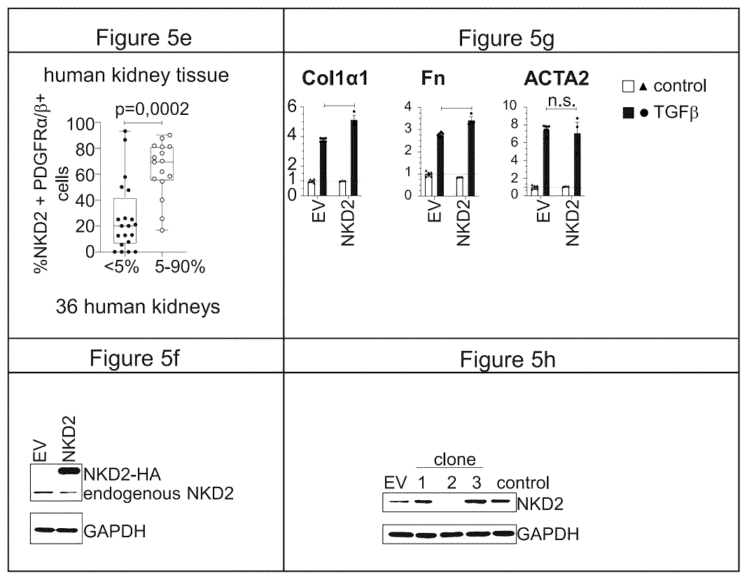

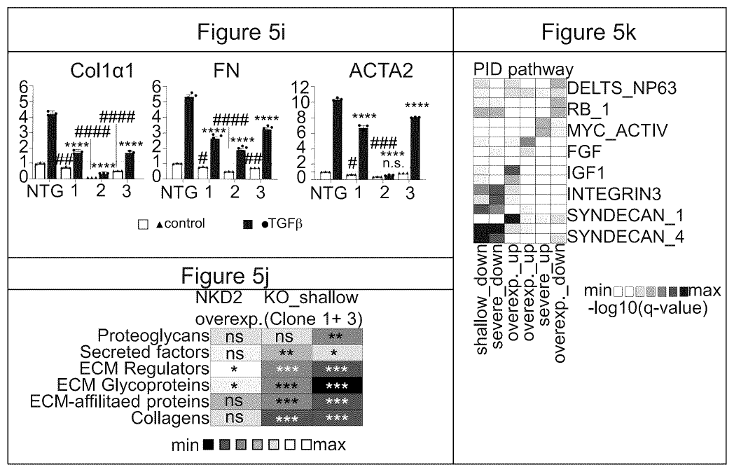

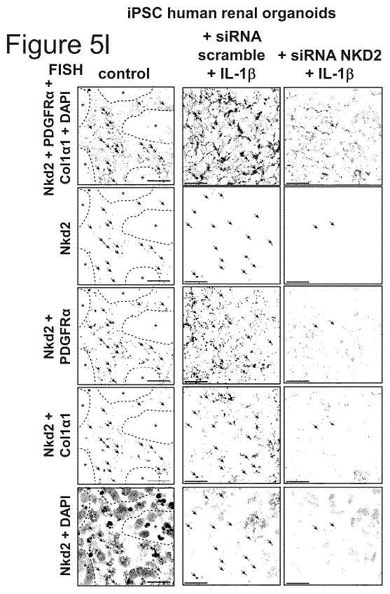

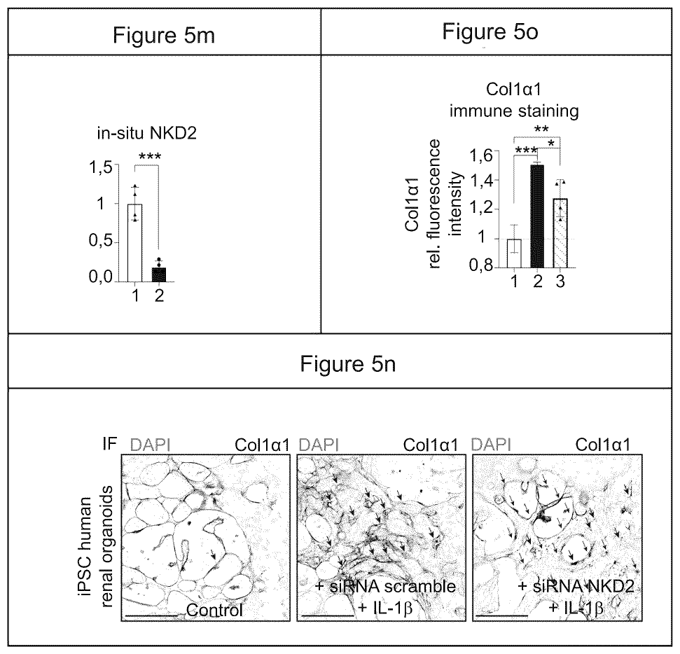

Scaled gene expression heatmaps such as those in FIG. 2d were produced using the plotMarkerHeat and plotTopMarkerHeat functions in the genesorteR R package. The fraction of expressed cells heatmaps such as FIG. 3d were produced using plotBinaryHeat function from the genesorteR R package. Heatmaps showing log 2-fold-changes and enrichments of features such as FIG. 5j,k were produced using Complex Heatmap R package (v. 2.4.2).

### ATAC-Seq Analysis

Illumina Tn5 adapter sequences were trimmed from ATAC-Seq reads using bbduk command from BBmap suite (version 38.32, settings: trimq=18, k=20, mink=5, hdist=2, hdist2=0). STAR (version2.7.0e) was used to map ATAC-Seq reads to the mm10 genome assembly retaining only uniquely mapped pairs (settings: alignEndsType EndToEnd, alignIntronMax 1, alignMatesGapMax 2000, alignEndsProtrude 100 ConcordantPair, outFilterMultimapNmax 1, outFilterScoreMinOverLread 0.9, outFilterMatchNminOverLread 0.9). Picard's MarkDuplicates command (version 2.18.27) was used to remove sequence duplicates (settings: remove_duplicates=TRUE, http://broadinstitute.github.io/picard/). Non-concordant read pairs were then removed from the BAM file using Samtools (version 1.3.1) 39. bedtools (version 2.17.0) was used to convert BAM files to BED files and to extend each read to 15 bp upstream and 22 bp downstream from the read 5′-end in a stranded manner 40, in order to account for steric hindrance of Tn5-DNA contacts 41.JAMM (version 1.0.7rev5) was used to identify open regions from the final BED files keeping the two replicates separate, retaining peaks that were at least 50 bp in width in the all list for further analysis (parameters: −r peak, −f 38,38, −e auto, −b 100)42. ATAC-Seq signal bigwig files were produced using JAMM SignalGenerator pipeline (settings: −f 38,38−n depth).

To deconvolute ATAC-Seq signal from bulk ATAC-Seq data according to scRNA-Seq clustering, we followed the following strategy. To deconvolute the ATAC-Seq signal three main steps in the data analysis were taken: 1) each open chromatin peak (where TFs are expected to bind DNA) was first assigned to a specific gene. 2) these genes were ranked per scRNA-Seq cluster (Fib, MF1/2 etc) depending on their expression in the single-cell RNA-Seq dataset. 3) The top 2000 ATAC peaks were used to identify enriched transcription factor motif sequences.

In more detail, each open chromatin ATAC-Seq peak was assigned to a gene according to its closest annotated transcription start site using the bedtools closest function, setting 100 kb as the maximum possible assignment distance. ATAC-Seq peak ranking per scRNA-Seq cluster was obtained by ranking the peaks according to the ranking of their assigned gene in the single cell RNA-Seq cluster. The top 2000 ATAC-Seq peaks for each scRNA-Seq cluster were selected and XXmotif was used for de novo motif finding for each scRNA-Seq cluster open chromatin regions separately (settings: —revcomp —merge-motif-threshold MEDIUM). We kept only motifs whose occurrence was more than 5%, as defined by XXmotif, for further analysis. Motif occurrence from all motifs from all 4 scRNA-Seq clusters were quantified using FIMO 44 with default parameters (MEME version 5.0.1) in the peaks assigned to the top 200 genes in each single cell RNA-Seq cluster. This produced a frequency matrix of motif occurrence in scRNA-Seq clusters. To quantify enrichment/depletion of motif occurrence in scRNA-Seq clusters we plot the log 2 fold-change of those frequencies relative to the average frequency obtained by randomizing the true frequency matrix 1000 times while keeping row and column sums constant. Randomization was performed using the R package Vegan (https://CRAN.R-project.org/package=vegan). Positive numbers indicate enrichment relative to what would be expected by chance, negative numbers indicate depletion (see Main FIG. 4k). We selected Irf8, Nrf,Creb5/Atf3, Elf/Ets and Klf for further investigation. We plotted the signal from all peaks that contained those motifs using DeepTools version 3.3.1, using the bigwig file generated by JAMM as input. We visualized the same bigwig file and motif occurrence in the Integrative Genomics Viewer.

### Other Visualization/Analysis

Heatmaps that do not quantify gene expression were produced using the heatmap2 function in the gplots R package (https://CRAN.R-project.org/package=gplots). Violin plots were produced using the vioplot R package (https://CRAN.R-project.org/package=vioplot).

### Quantification and Statistical Analysis Used Outside of the Single Cell Sequencing Data

Data are presented as mean±SEM if not specified otherwise in the legends. Comparison of two groups was performed using unpaired t-test. For multiple group comparison one-way ANOVA with Bonferroni's multiple comparison test was applied or two-way ANOVA with Sidak's multiple comparisons test. Statistical analyses were performed using GraphPad Prism 8 (GraphPad Software Inc., San Diego, CA). A p-value of less than 0.05 was considered significant.

### Gene Regulatory Network Analysis

Gene expression was 11-scaled per gene and the pearson correlation coefficient was calculated between Nkd2 and all other genes along pericyte, fibroblast and myofibroblast single cells. The top 100 correlating and top 100 anti-correlating genes were selected for pathway enrichment analysis. Further the expression of those 200 genes along single cells was used as input to GRNboost2+ python package to predict putative regulatory links between genes. The output network was filtered by removing connections with strength <=10. The resulting network was plotted as an undirected network (since regulators are not known beforehand) using ggraph package (https://cran.r-project.org/web/packages/ggraph/index.html) and clustered into 4 modules using the Louvain algorithm as implemented in the igraph package.

Transcription Factor Predictions from Single Cell Data

To obtain transcription factor scores in distal and proximal regions, we used the top 200 marker genes for fibroblast, pericyte and myofibroblast cell clusters as input gene lists to RCisTarget. We followed the RCisTarget Vignette to perform the analysis with default parameters (available https://bioconductor.org/packages/release/bioc/vignettes/RcisTarget/inst/doc/RcisTarget.html). To quantify AP1 expression, we used all Jun and Fos genes as a geneset and applied the same method to obtain an AP1 score as we did for ECM score. To quantify AP1 activity (defined as the expression of putative target genes, we defined AP1 target genes according to the Dorothea regulon database and applied the same method as ECM score to obtain a single cell AP1 activity score.

### Mouse Supervised Cell Classification

We classified single cells in the mouse PDGFRa+b+ dataset using the human PDGFRb+ dataset as a reference using the CHETAH algorithm with default parameters. Human gene symbols were converted to mouse gene symbols using the biomaRt database.

### CellphoneDB Analysis

CellPhoneDB (v.2.1.1) was used to estimate cell-cell interactions among the cell types found in the human CD10-fraction using the version 2.0.0 of the database, and the normalized gene expression as input, with default parameters (10% of cells expressing the ligand/receptor). Interactions with p-value <0.05 were considered significant. We consider only ligand-receptor interactions based on the annotation from the database, for which only and at least one partner of the interacting pair was a receptor, thus discarding receptor-receptor and other interactions without a clear receptor. Ligand-receptor interactions from pathways involved in kidney fibrosis were selected using the membership from KEGG database for Hedgehog, Notch, TGFb and WNT signaling, and REACTOME database for EGFR signaling from MSigDB 3, and manual curation for PDGF signaling.

### Bulk RNA-Seg Data Analysis

Gene expression was quantified on the transcript level using Salmon v1.1.0, with the —validatMappings and —gcBias parameters switched on, to the human Gencode v29 transcriptome. Transcript level counts were aggregated to gene level counts using the import in tximport R package, setting countsFromAbundance to “lengthScaledTPM”. Limma R package (v.3.44.1) was used to test for differential gene expression between Nkd2-perturbed human kidney PDGFRb+ as compared to their control using the empirical Bayes method after voom transformation. We found that two out of the three clones of CRISPR-Cas9 NKD2 Knock-Out group together in the principal component analysis and exhibited a shallow phenotype, while the third clone grouped independently and presented a more severe phenotype. Thus, we grouped the two first clone knock-outs, to have two independent Knock-Out conditions for the statistical contrasts. Differentially expressed genes were ranked by the moderated t-statistic from the statistical test for pathway and gene ontology analysis. P-values were adjusted for multiple testing using Benjamini & Hochberg method. Genes and pathways with FDR <0.05 were considered significant.

For pathway and gene ontology analysis, we also used clusterProfiler R package with KEGG and PID pathways using genes with adjusted p-value less than 0.01 in the Nkd2-perturbed cells as compared to the control and absolute log fold-change higher than 1 for knockout comparison (higher than 0 for over-expression comparison) with a maximum of 200 genes, ranked by the adjusted p-value. We used GSEA-preranked to test for an enrichment of ECM genes in the phenotypes using fgsea R package (v.1.14.0) 54, with MatrisomeDB gene set collection.

### Example 2: Single Cell Atlas of Human Chronic Kidney Disease

To understand which resident cell types in the human kidney secrete extracellular matrix during homeostasis and CKD, we generated a single cell map of human kidneys with a particular focus on the tubulointerstitium. Over 80% of renal cortical cells are proximal tubule epithelial cells and as such have tended to dominate previous single cell maps of the kidney, masking potentially important heterogeneity in other renal cellular compartments. We therefore chose a sorting strategy that enriches for live (viable), non-proximal tubule epithelial cells (i.e. cells negative for CD10, also known as membrane metallo-endopeptidase-MME) but also sorted the live, CD10+ proximal tubule epithelial fraction to map the entire kidney in an unbiased fashion. Of note, this is a non-exclusive sorting strategy since CD10 is also expressed by some other cell types. However, it allows an enrichment/depletion of proximal tubule epithelial cells. Both CD10+ and CD10-fractions from a total of 13 patients with different stages of CKD due to hypertension induced nephrosclerosis (n=7; estimated Glomerular Filtration Rate, eGFR>60 and n=6; eGFR<60) were subjected to scRNAseq. We profiled 53,672 CD10-cells from 11 patients, (n=7 eGFR>60; n=4 eGFR<60). Patients with eGFR<60 showed increased interstitial fibrosis and tubular atrophy. To integrate the data across patients, we employed an unsupervised graph-based clustering method (see Methods) and identified 50 different CD10-cell clusters (FIGS. 1a-d) represented in both eGFR groups (FIG. 1e). Our sorting and data integration strategies allowed us to appreciate the full scale of heterogeneity in the renal interstitium including identification of rare cell types such as Schwann cells that have not been described in previous kidney single cell maps (FIGS. 1a-d).

A total of 33,690 CD10+ proximal tubule cells were profiled (from 8 patients (n=5; eGFR>60 and n=3; eGFR<60) and arranged into 7 clusters (FIG. 1f). Cell-cycle analysis of the CD10+ proximal tubule cell clusters indicated increased cycling in CKD likely reflecting an epithelial repair response (FIG. 1g). KEGG pathway analysis and Gene Ontology terms in CD10+ cells suggested increased fatty acid metabolism among various other dysregulated metabolic pathways in CKD (FIG. 1h). Fatty acid metabolism has been reported as a key dysregulated pathway in human and mouse kidneys causing tubular dedifferentiation and fibrosis (Kang et al 2015).

Thus, we employed a sorting strategy that generated a high resolution map of human kidneys in homeostasis and CKD to allow the subsequent interrogation of the cellular origin of ECM during human CKD.

### Example 3: Origin of Extracellular Matrix in Human Chronic Kidney Disease

To understand which cell types contribute to extracellular matrix (ECM) production during progression of human kidney fibrosis, we established a single cell ECM expression score summarizing the expression of all core ECM molecules including collagens, glycoproteins and proteoglycans. We validated this score in a published dataset of 36 patients with diabetic nephropathy (Fan et al 2019), confirming increased ECM score values in advanced CKD.

ECM scores demonstrated a clear shift towards high ECM expressing cells in CKD (FIG. 1i). We then compared the ECM score of the major cell types in homeostasis and CKD, identifying mesenchymal cells as the cells with the highest ECM expression and a further increase in ECM expression in CKD. Whilst we did not observe significantly increased expression of ECM in any of the mesenchymal subclusters in CKD, all fibroblast and myofibroblast populations expanded in CKD explaining the overall increased ECM gene expression observed in the mesenchyme (FIG. 1k-1). Historically, ACTA2 was used as a myofibroblast marker. However, since ECM expression is the hallmark of fibrosis, we have defined myofibroblasts as cells that express most ECM genes. Beside expansion of individual cells another important mechanism of increased ECM expression is differentiation of cells into the ECM high myofibroblasts. To further investigate the heterogeneity of ECM-expressing mesenchymal populations and putative differentiation processes in human CKD, we generated a Uniform Manifold Approximation and Projection (UMAP) embedding of (myo) fibroblasts and pericytes (FIG. 1m-n). This UMAP embedding was in agreement with our unsupervised graph clustering results (FIG. 1b-c), and highlights the previously underappreciated heterogeneity of the human renal mesenchyme. Myofibroblasts were clearly identified as periostin (postn) expressing cell clusters (FIG. 1n). Diffusion mapping is a dimensionality reduction method that assumes that cells relate to each other by a differentiation-like diffusion process. We used this method to unravel putative differentiation mechanisms towards myofibroblasts. A diffusion map embedding of mesenchymal cells with the highest ECM expression levels suggested that myofibroblasts arise from pericytes and fibroblasts (FIG. 10).

We observed a minor upregulation of ECM genes in epithelial cells (FIG. 1j), suggestive of a minor contribution of epithelial mesenchymal transition (EMT), which has been debated in the kidney community for many years. Injured proximal tubule epithelium (iPT) showed the highest expression of ECM genes among CD10-epithelium with various expressed genes and GO terms suggesting de-differentiation from regular epithelium. In the CD10+ fraction (all sorted proximal tubule epithelium), we also observed a slight increase in ECM expression in CKD. Of note, injured cells were defined by expression of genes previously reported as injury-related such as Sox9, CD24 and CD133 for proximal tubule epithelium and VCAM1 and ACKR1 for endothelium.

In summary, these data indicate that the vast majority of ECM generated during human kidney fibrosis originates from multiple different mesenchymal cell subtypes, with only a minor contribution from dedifferentiated tubular epithelial cells.

### Example 4: Distinct Pericyte and Fibroblast Subpopulations are the Major Source of Myofibroblasts in Human Kidney Fibrosis

Our CD10-scRNA-seq data indicated that the vast majority of Col1a1 expressing cells are PDGFRb+. We therefore sorted 37,380 PDGFRb+ cells from 8 human kidneys (n=4; eGFR>60 and n=4; eGFR<60). Unsupervised clustering identified mesenchymal cell populations and some epithelial, endothelial and immune cells (FIG. 2a-d), which were annotated according to their correlation with the CD10-populations. Collagen, and ECM gene expression in general, was dominant in the pericyte, fibroblast and myofibroblast clusters, in agreement with our CD10-data (FIG. 1j-k). However, some macrophage/monocyte, endothelial and injured epithelial populations also expressed collagen1a1 and PDGFRb, but at much lower levels than mesenchymal cells (FIG. 2a-c). Computational predictions of doublet-likelihood scores did not show particularly high scores for endothelial and injured epithelial cells, however, the score was slightly increased for the macrophage population. We verified Col1a1 mRNA expression in LTA+ proximal tubule, CD68+ macrophages and Pecam-1+ endothelial cells by in situ hybridisation (ISH). These data may to some degree explain the controversy in the literature regarding the contributions of non-mesenchymal lineages to the renal myofibroblast pool (Duffield 2014; Wang et al 2017) since we observed indeed minor ECM gene expression in these non-mesenchymal cell types, whilst the majority of ECM gene expression is mesenchymal cell-derived.

Pseudotime trajectory and diffusion map analysis of the major ECM expressing cellular subtypes from the PDGFRb+ populations indicated three major sources of myofibroblasts in human kidneys: 1) Notch3+/RGS5+/PDGFRa-pericytes, 2) Meg3+/PDGFRa+ fibroblasts and 3) Colec11+/CXCL12+ fibroblasts (FIG. 2e). Of note, diffusion mapping places non-CKD cells mainly within the low ECM-expressing pericyte and fibroblast populations, indicating a potential differentiation trajectory from low-ECM, non-CKD mesenchymal cells (pericytes and fibroblasts) to high-ECM CKD myofibroblasts (FIG. 2e). A UMAP embedding of these mesenchymal cells was also consistent with these results. This is consistent with a differentiation towards myofibroblasts that express ECM and Postn in kidney fibrosis. We verified this predicted directionality using ISH in human kidneys, confirming increased numbers of Postn expressing cells in kidney fibrosis (FIG. 2f), whilst the number of Meg3+ cells decreased. Using ISH we further validated the main lineages in our diffusion map analysis, consisting of Notch3+ pericytes (lineage 1) and Meg3+ fibroblasts (lineage 2) (FIG. 2f). We observed both Meg3+ and Notch3+ cells co-expressing Postn indicating myofibroblast differentiation (FIG. 2f). Interestingly, we also observed a likely intermediate stage of Notch3/Meg3/Postn co-expressing cells which may represent differentiating cells in the center of the diffusion map (FIG. 2f). We then assessed whether the identified mesenchymal subpopulations are also spatially distinct. Whilst we did not observe distinct spatial localisation for fibroblasts 1 (Meg3+), pericytes (Notch3+) or fibroblasts 2 (Cxcl12+), we observed myofibroblasts 1 (Postn+) cells enriched in areas of fibrosis as expected. Interestingly, myofibroblasts 3 (Cc119+/Ccl21+), which increases in human kidney fibrosis, exhibited distinct enrichment around glomeruli.

We then analysed the gene expression program of pericyte-to-myofibroblast differentiation (Lineage 1) (FIG. 2g). Cell cycle analysis demonstrated profound changes, consistent with both a differentiation process and expansion of the myofibroblast population (FIG. 2g). To better understand pericyte-to-myofibroblast differentiation, we ordered pathway enrichment along pseudotime. We observed early (canonical Wnt, Myc and AP1), intermediate (ATF2, PDGFRa and Myc) and late (integrin, ECM receptor interaction and TGFb) signaling among other pathways (FIG. 2g bottom).

Similar to pericyte-to-myofibroblast differentiation, we observed cell cycle cessation during fibroblast-to-myofibroblast differentiation, followed by increased proliferation (lineages 2 and 3). Pathway enrichment ordered along pseudotime highlighted early AP1 signaling, inflammatory and immune cell interaction pathways, which were followed by integrin signaling, focal adhesion and ECM interaction pathways.

TGFb signaling was prevalent in the pseudotime analysis of lineage 2 (FIG. 2g). Myofibroblasts 1, which likely represent fully differentiated myofibroblasts, expressed high levels of TGFb ligands and lower levels of TGFb receptors. However, the opposite was observed for fibroblasts 1, suggesting a mechanism whereby myofibroblasts may promote differentiation of fibroblasts.

Many of the pathways described above are known to be important regulators of fibrosis, including integrins27 and also AP1 transcription factor signaling (Wernig et al 2017) which we observed were consistently highly active during the early stages of both pericyte- and fibroblast to myofibroblast differentiation. To further understand transcriptional regulation of fibroblast and myofibroblast populations, we performed transcription factor DNA sequence motif enrichment analysis in promoters and distal regions of marker genes in various mesenchymal populations. This further highlighted a potential key regulatory role of AP-1 (Jun/Fos) in fibroblast to myofibroblast differentiation. To functionally validate the role of AP1, we generated a novel human PDGFRb+ kidney cell line using lentiviral hTERT, SV40LT transduction. Pharmacological inhibition of activator-protein 1 (AP1) resulted in significantly decreased proliferation and decreased osteoglycin (Ogn) expression, whilst Postn expression was increased, suggesting myofibroblast differentiation of these cells. Of note, in the human PDGFRb data, Ogn marked fibroblast 1/3 while Postn marked myofibroblasts 1. Consistent with these results, AP1 expression correlates negatively with collagen expression in both fibroblasts and myofibroblasts. Interestingly, the expression of AP1's putative target genes positively correlated with collagen expression in myofibroblasts, indicating that AP1 might act as a suppressor. We also performed ligand-receptor analyses (Efremova et al 2020) to help elucidate which cell types interact with the key ECM expressing mesenchymal cells (fibroblasts, pericytes and myofibroblasts). While we observed that the least signaling came from healthy proximal tubule epithelium, injured proximal tubule epithelium was among the top signaling partners to the mesenchyme, in line with tubule-interstitial signaling as a hallmark of kidney fibrosis (Venkatachalam et al 2015). We focused on interactions from pathways that have been described to be key players in fibrosis including TGFb, PDGFRa/b, Notch, EGFR and WNT signaling (Kramann and DiRocco 2013). Within these pathways we observed Notch, TGFb, Wnt and PDGFa signaling from the injured proximal tubule towards the mesenchymal fibrosis driving cells.

In summary, these data suggest three main cellular sources of human kidney myofibroblasts, that are all marked by PDGFRb, namely Notch3+/PDGFRa− pericytes, Meg3+/PDGFRa+ fibroblasts, and Colec11+/Cxcl12+ fibroblasts and sheds light into their differentiation processes.

### Example 5: Dual-Positive PDGFRa+/PDGFRb+ Mesenchymal Cells Represent the Majority of ECM-Expressing Cells in Human and Mouse Kidney Fibrosis

We used genetic fate tracing in mice to further interrogate the findings presented above. PDGFRbCreER-tdTomato mice were pulsed with tamoxifen and subjected to unilateral ureteral obstruction (UUO) surgery and sacrificed at day 10 (FIG. 3a). In situ hybridization (ISH) for Col1a1 mRNA confirmed that virtually all Col1a1 expressing cells in mouse kidney fibrosis are PDGFRb lineage derived (FIG. 3b-c). Furthermore, immunostaining for the historically used myofibroblast marker aSMA (ACTA2) confirmed that the majority of aSMA expressing cells were PDGFRb lineage derived. We then performed a SmartSeq2-based sc-RNA-seq time-course study in PDGFRb-eGFP mice (Picelli et al 2014) (FIG. 3d-e). While smooth muscle cells and pericytes decrease in abundance over time following UUO, mesangial cells and Col1a1+/PDGFRa+ matrix producing cell clusters increased considerably over time (FIG. 3f-g). Similar to our human kidney datasets (FIGS. 1,2), the major ECM-expressing cell population was defined by dual PDGFRa/PDGFRb expression and also expression of decorin (DCN) and periostin (Postn) (FIG. 3g-h). Furthermore, pericytes and vascular smooth muscle cells (vSMCs) showed some ECM expression, but at a significantly lower level than the dual PDGFRa/PDGFRb population.

Immunostaining and ISH in mice confirmed that Col1a1-expressing cells are double positive for PDGFRa+ and PDGFRb-tdTomato (FIG. 3i-j). This is in agreement with our human CD10-data where selection for PDGFRa+/b+ expressing cells enriched for Col1a1+ cells, identifying PDGFRa/PDGFRb expressing cells as the major source of ECM expression (FIG. 3k). We confirmed this finding in a larger human cohort using multiplex ISH in tissue microarrays of 62 patients (FIG. 3l). Diffusion map embedding of matrix producing cells and pericytes was also in line with our human PDGFRb data, and suggested that pericytes (PDGFRb+, PDGFRa−, Notch3+) are one of the origins of the major ECM-producing cells (PDGFRb+, PDGFRa+, Colla1+, Postn+).

Taken together, our human data and fate-tracing experiments in mice demonstrate that PDGFRa+/PDGFRb+ dual-positive mesenchymal cells, which include all fibroblast and myofibroblast populations, including pericyte-derived myofibroblasts but not non-activated pericytes (i.e. pericytes that do not exhibit high ECM gene expression) (FIG. 2e), represent the majority of Col1a1 expressing cells in both human and mouse kidney fibrosis.

### Example 6: PDGFRa+/PDGFRb+ Cells are Heterogeneous and Contain Different Fibroblast Cell States

To gain mechanistic insights into the transition of fibroblasts to myofibroblasts and dissect the heterogeneity of the PDGFRa+/PDGFRb+ population, we next generated scRNA-Seq data from 7,245 dual positive PDGFRa+/PDGFRb+ mouse kidney cells by performing UUO surgery versus sham in PDGFRb-eGFP mice, followed by sorting of eGFP/PDGFRa double positive cells (FIG. 4a). Consistent with a rapidly expanding cell population, the PDGFRa+/PDGFRb+ double positive cells showed a ˜140-fold increase in cell numbers after injury (FIG. 4b), in agreement with our Smart-Seq2 data (FIG. 3f). UMAP embedding of PDGFRa+/PDGFRb+ cells revealed four major, distinct populations corresponding to mesenchyme (fibroblasts and myofibroblasts), epithelial, endothelial and immune cells (FIG. 4c-d). All these cell types have previously been discussed as a potential cellular origin of kidney fibrosis (Duffield et al 2014; Wang et al 2017; Kramann et al 2018). Of note, we did not detect any undifferentiated pericytes in this PDGFRa+/PDGFRb+ data, since pericytes are PDGFRa− in humans and mice (FIG. 2e, 3g). Non-mesenchymal cells expressed markedly lower PDGFRb, PDGFRa, ECM and collagen levels than mesenchymal cells (FIG. 4d-e), supporting the observation in our human data that non-mesenchymal cells are minor contributors to the scarring process (FIG. 1,2). Of note, as in the human data, computationally-derived doublet scores do not suggest that these matrix-expressing non-mesenchymal cell populations are likely doublets.

Unsupervised clustering revealed two key classes within mesenchymal cells in this mouse PDGFRa+/PDGFRb+ dataset (1) fibroblast 1 marked by Scara5 and Meg3 expression and (2) myofibroblasts consisting of various myofibroblast subpopulations (FIG. 4c-d). In our human data, myofibroblasts 1 correspond to terminally differentiated myofibroblasts with the highest ECM expression preceded in differentiation pseudotime by myofibroblast 2 (Ogn+), while fibroblasts 1 appeared as a “progenitor” non-activated fibroblast population (FIG. 2e). Indeed, fibroblast 1 cells can be distinguished from myofibroblasts in the PDGFRa+/PDGFRb+ data by three major features: First, Col15a1, a myofibroblast-specific collagen in mice (FIG. 3g), was expressed at lower levels in fibroblasts 1 than the myofibroblast clusters (FIG. 4f). Second, although Meg3 is also expressed in a fraction of proximal tubular cells and glomerular endothelium, Meg3 was only detected in fibroblasts 1 within the mesenchymal populations (FIG. 4d). We validated the presence of a Meg3+PDGFRa+/PDGFRb+ mesenchymal subpopulation in human kidneys by in situ hybridization (FIG. 4h-i), suggesting the presence of a fibroblast 1-like subpopulation in human kidneys. Third, fibroblast 1 cells are Scara5+ but Frzb−, again demonstrating that they are distinct from myofibroblasts.

Having established fibroblasts 1 as a distinct fibroblast population, we generated UMAP and diffusion map embeddings and performed pseudotime analyses of all mouse Pdgfra+/Pdgfrb+ mesenchymal cells to gain insight into their lineage relationships (FIG. 4j). This analysis suggested fibroblast 1 (Meg3+, Scara5+) and myofibroblast 2 (Col14a1+, Ogn+) as early states, myofibroblast 3a as an intermediate state, and myofibroblast 1a (Nrp3+, Nkd2+), 1b (Grem2+) and 3b (Frzb+) as terminal states (FIG. 4j).

These data suggest that fibroblasts 1 and myofibroblasts 2 are the major source of myofibroblasts in mouse kidney fibrosis. Myofibroblasts 2 (Ogn+/Col14a1+) might exist in healthy mouse kidneys or may arise as an intermediate state due to pericyte to myofibroblasts differentiation (FIG. 2e, human data). Angiotensin receptor 1 (AGTR1a) expression is enriched in myofibroblasts 2 and might point towards their pericyte origin (FIG. 4j).

Analysis of the time-course UUO data shows Ogn, Scara5 and Pcolce2 as being enriched in homeostasis, while naked cuticle homolog 2 (Nkd2) is enriched after injury. This further suggests fibroblast 1 (Meg3+, Scara5+) and myofibroblasts 2 (Ogn+) as cells present in kidney homeostasis. Furthermore, supervised classification of the mouse Pdgfra+/Pdgfrb+ single cell data using our human Pdgfrb+ cells as a reference confirms the distinct identity of fibroblasts 1 and myofibroblasts as a common feature in both species.

Overall, our combined and comprehensive human and mouse data suggest a model in which Pdgfrb+/Pdgfra+/Postn+ high-ECM expressing myofibroblasts (termed myofibroblast 1 throughout this manuscript) arise from Pdgfrb+/Pdgfra-/Notch3+ pericytes, Pdgfrb+/Pdgfra+/Scara5+ fibroblasts (fibroblasts 1) and Pdgfrb+/Pdgfra+/Cxcl12+ fibroblasts (fibroblasts 2). Pericytes differentiate potentially through an intermediate ECM-expressing Pdgfrb+/Pdgfra+/Ogn+/Col14a1+ (myofibroblasts 2) state into myofibroblasts 1.

### Example 7: Distinct Fibroblast and Myofibroblast Cell States are Distinguished by Specific Transcription Factor Regulatory Programs

Next, we sought to ascertain whether fibroblast and myofibroblast cell states detected in our data represent truly distinct cell types. Distinct cell types would be distinguished by both distinct gene expression profiles and distinct transcription factor regulatory programs (Gerstein et al 2012). We generated bulk ATAC-Seq (Buenrostro et al 2013) data from Pdgfra+/Pdgfrb+ mouse kidney cells 10 days after UUO surgery, and deconvoluted the open chromatin region (OCR) signatures from ATAC-Seq data based on OCR proximity to marker genes identified in the scRNA-Seq clusters. Fibroblasts 1 and myofibroblasts 2 were both distinct from each other and from other myofibroblast populations. Myofibroblasts 1a were distinct from myofibroblasts 1b and featured enrichment of ATF. Myofibroblasts 2 and 3b showed enrichment of the orphan receptor NRF4A1 which has been previously reported as an important regulator of TGFb signaling and fibrosis (Palumbo-Zerr et al 2015). Fibroblasts 1 showed enrichment of AP-1 (Jun/Fos) motifs (FIG. 4k), consistent with their putative role outlined in our human data. RNA expression of these ATAC-Seq selected factors is in line with sequence motif enrichment (FIG. 4k) and highlights the divergent transcriptional regulation between fibroblasts 1, myofibroblasts 2 and other myofibroblast populations. We further highlight transcription factors that might be underappreciated in relation to kidney fibrosis including Nrf1, Irf8 and Creb5. Congruent with our ATAC-Seq data, signaling pathway analysis based on our scRNA-Seq data indicated that fibroblasts 1 and myofibroblasts are distinct populations with different enriched pathways (FIG. 4l). Therefore, fibroblasts 1 and myofibroblast subtypes are likely distinct ECM-expressing mesenchymal cell types, harboring specific transcription factor regulatory programs.

### Example 8: Nkd2 is Required for Collagen Expression in Human Kidney PDGFRb+ Cells and is a Potential Therapeutic Target in Kidney Fibrosis

We next asked whether the scRNA-seq data we have generated could be used to identify potential therapeutic targets in human kidney fibrosis. Nkd2 is specifically expressed in mouse Pdgfra+/Pdgfrb+ terminally differentiated myofibroblasts (FIG. 5a), such that Nkd2/PDGFRa dual positive cells constituted >40% of all Col1a1+ cells (FIG. 5b). In human PDGFRb+ cells, NKD2 is a marker of high ECM myofibroblasts where its expression positively correlates with Postn and ECM expression and anti-correlates with genes associated with pericytes and fibroblasts (FIG. 5c). In addition, NKD2+ myofibroblasts were associated with increased TGFb, Wnt and TNFα pathway activity compared to NKD2-cells. We verified NKD2 expression by multiplex ISH in a human kidney tissue microarray (TMA) of 36 patients, confirming that a subpopulation of human PDGFRa/PDGFRb expressing cells also expresses Nkd2 (FIG. 5d-e). Furthermore, the abundance of PDGFRa/PDGFRb/Nkd2 co-expressing cells was higher in patients with more pronounced interstitial fibrosis (FIG. 5e).

Nkd2 has been documented as a Wnt pathway and TNFα modulator (Zhao et al 2015; Hu and Li 2010; Hu et al 2010; Li et al 2004). In order to understand the mechanisms by which Nkd2 regulates kidney fibrosis, we used our human PDGFRb+ data to predict a gene regulatory network focused on genes correlated with Nkd2, using the GRNboost2 framework. The resulting network clustered into 4 gene regulatory modules including ribosomal proteins (module 1), genes related to ECM expression (module 2), genes related to pericytes (module 3) and genes related to non-activated fibroblasts (module 4). Of note, this gene cluster included various Wnt modulators and effectors in addition to Nkd2 such as Kif26b, Lef1 and Wnt4. Nkd2 is placed with ECM genes and is connected to Etv1 and Lamp5, and indirectly to Col1a1 through Lamp5. This analysis may suggest a potential mechanism by which Nkd2 is regulated by Etv1 (a member of the Ets factor family), and acts by affecting paracrine signaling through Lamp5.

Lentiviral overexpression of Nkd2 in our human PDGFRb cell line resulted in increased expression of key pro-fibrotic ECM molecules such as col1a1 and fibronectin in response to TGFb (FIG. 5f-g). Importantly, CRISPR/Cas9 knockout of Nkd2 resulted in a marked reduction in col1a1, fibronectin and ACTA2 expression in the presence or absence of TGFb (FIG. 5h-i). RNA-seq from cells overexpressing Nkd2 demonstrated upregulation of ECM regulators and ECM glycoproteins, whilst RNA-seq of Nkd2 knockout clones indicated a loss of ECM regulators, ECM glycoproteins and collagens (FIG. 5j). Pathway and Gene Ontology analysis demonstrated a role for Nkd2 in ECM expression programs and suggested further interplay with AP1 and integrin signaling pathways (FIG. 5k). We further observed strong changes in the expression of Wnt receptors and ligands following Nkd2 knockout in vitro confirming its potential involvement in this pathway.

To further validate Nkd2 as a therapeutic target, we generated induced pluripotent stem cell (iPSC) derived kidney organoids which contain all major compartments of the human kidney. IL1b is well established to induce fibrosis in iPSC derived kidney organoids (Lemos et al 2018). Importantly, siRNA mediated knockdown of Nkd2 inhibited IL1b-induced Col1a1 expression in the kidney organoids (FIG. 5l-o). These data confirm that Nkd2 marks myofibroblasts in human and mouse kidney fibrosis, is required for renal myofibroblast collagen expression, and therefore represents a promising potential therapeutic target to treat patients with kidney fibrosis.

### Example 9: Screening for Agents Binding to and/or Inhibiting NDK2 Protein

Screening experiments allow for identification and validation of small-molecule therapeutic compounds, peptides and/or biologics that bind and/or inhibit the activity of NKD2 protein.

DNA-bar coded compound libraries are generated and screened as described (Kunig et al. 2018). To this end, recombinant NKD2 protein, or fragments thereof, carrying a His tag, are expressed in E. coli, insect cells or mammalian cells. Purified NKD2 protein is incubated with the compound library and isolated by immunoprecipitation. The compounds bound to NKD2 protein are identified by way of Sanger sequencing of the DNA bar codes. The identified compounds are subsequently tested for effects on the function of NKD2, the differentiation of myofibroblast cells, the expression and secretion of matrix proteins, such as for example collagen 1, and the development of kidney fibrosis. To this end, experimental mouse in-vivo models of kidney fibrosis are employed.

For identification and validation of small-molecule therapeutic compounds, peptides and/or biologics exerting an effect on nkd2 expression, an in-vitro human cell-based fluorochrome reporter system is established, using for example eGFP NKD2 fusion protein expression or luciferase-based reporter system in order to screen compound libraries in 384- to 1,536-well-format assays for identification of compounds reducing eGFP fluorescence or luciferase levels as readout. Expression of these human NKD2 fusion reporter constructs in said cells can be performed, e.g., by transfection and selection via resistance gene cassettes, or by viral transduction. For these assays, human cell lines like, e.g., 293T cells, but also established human kidney myofibroblast cell lines are employed. In parallel to this screening, cytotoxicity assays are performed in order to exclude compounds exerting an effect on the reporter fluorescence or activity due to unspecific toxicity or triggering of apoptosis.

### REFERENCES

Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
Coifman, R. R. et al. Geometric diffusions as a tool for harmonic analysis and structure definition of data: diffusion maps. Proc. Natl. Acad. Sci. U.S.A. 102, 7426-7431 (2005).
Djudjai, S. & Boor, P. Cellular and molecular mechanisms of kidney fibrosis. Mol. Aspects Med. 65, 16-36 (2019)
Dobie, R. & Henderson, N. C. Unravelling fibrosis using single-cell transcriptomics. Curr. Opin. Pharmacol. 49, 71-75 (2019).
Duffield, J. S. Cellular and molecular mechanisms in kidney fibrosis. J. Clin. Invest. 124, 2299-2306 (2014).
Efremova, M., Vento-Tormo, M., Teichmann, S. A. & Vento-Tormo, R. CellPhoneDB: inferring cell-cell communication from combined expression of multi-subunit ligand-receptor complexes. Nat. Protoc. 15, 1484-1506 (2020).
Elices, M. J. et al. VCAM-1 on activated endothelium interacts with the leukocyte integrin VLA-4 at a site distinct from the VLA-4/fibronectin binding site. Cell 60, 577-584 (1990).
Falke, L. L., Gholizadeh, S., Goldschmeding, R., Kok, R. J. & Nguyen, T. Q. Diverse origins of the myofibroblast-implications for kidney fibrosis. Nat. Rev. Nephrol. 11, 233-244 (2015).
Fan, Y. et al. Comparison of Kidney Transcriptomic Profiles of Early and Advanced Diabetic Nephropathy Reveals Potential New Mechanisms for Disease Progression. Diabetes 68, 2301-2314 (2019).
Farber, D. L. & Sims, P. A. Dissecting lung development and fibrosis at single-cell resolution. Genome Med. 11, 33 (2019).
Friedman, S. L., Sheppard, D., Duffield, J. S. & Violette, S. Therapy for fibrotic diseases: nearing the starting line. Sci. Transl. Med. 5, 167sr1 (2013).
Gerstein, M. B. et al. Architecture of the human regulatory network derived from ENCODE data. Nature 489, 91-100 (2012).
Gotze S. et al. Frequent promoter hypermethylation of Wnt pathway inhibitor genes in malignant astrocytic gliomas. International journal of cancer. 126, 2584-2593 (2010).
Habermann, A. C. et al. Single-cell RNA-sequencing reveals profibrotic roles of distinct epithelial and mesenchymal lineages in pulmonary fibrosis. bioRxiv 753806 (2019) doi: 10.1101/753806.
Haghverdi, L., Buettner, F. & Theis, F. J. Diffusion maps for high-dimensional single-cell analysis of differentiation data. Bioinformatics 31, 2989-2998 (2015).
Henderson, N. C. et al. Targeting of αv integrin identifies a core molecular pathway that regulates fibrosis in several organs. Nat. Med. 19, 1617-1624 (2013).
Hu, T. & Li, C. Convergence between Wnt-β-catenin and EGFR signaling in cancer. Mol. Cancer 9, 236 (2010).
Hu, T. et al. Myristoylated Naked2 antagonizes Wnt-beta-catenin activity by degrading Dishevelled-1 at the plasma membrane. J. Biol. Chem. 285, 13561-13568 (2010).
Hu T. et al. Structural studies of human Naked2: a biologically active intrinsically unstructured protein. Biochemical and biophysical research communications. 350, 911-915 (2006).
Huang, S. & Susztak, K. Epithelial Plasticity versus EMT in Kidney Fibrosis. Trends in molecular medicine vol. 22 4-6 (2016).
Kang, H. M. et al. Defective fatty acid oxidation in renal tubular epithelial cells has a key role in kidney fibrosis development. Nat. Med. 21, 37-46 (2015).
Kang, H. M. et al. Sox9-Positive Progenitor Cells Play a Key Role in Renal Tubule Epithelial Regeneration in Mice. Cell Rep. 14, 861-871 (2016).
Kramann, R. & DiRocco, D. P. Understanding the origin, activation and regulation of matrix-producing myofibroblasts for treatment of fibrotic disease. J. Pathol. 231, 273-289 (2013).
Kramann, R. et al. Parabiosis and single-cell RNA sequencing reveal a limited contribution of monocytes to myofibroblasts in kidney fibrosis. JCI Insight 3, (2018).
Kriz, W., Kaissling, B. & Le Hir, M. Epithelial-mesenchymal transition (EMT) in kidney fibrosis: fact or fantasy? J. Clin. Invest. 121, 468-474 (2011).
Kunig, V. et al. DNA-encoded libraries—an efficient small molecule discovery technology for the biomedical sciences. Biol. Chem. 399 (7), 691-710 (2018).
Lake, B. B. et al. A single-nucleus RNA-sequencing pipeline to decipher the molecular anatomy and pathophysiology of human kidneys. Nat. Commun. 10, 2832 (2019).
Lemos, D. R. et al. Interleukin-1β Activates a MYC-Dependent Metabolic Switch in Kidney Stromal Cells Necessary for Progressive Tubulointerstitial Fibrosis. J. Am. Soc. Nephrol. 29, 1690-1705 (2018).
Li, C. et al. Myristoylated Naked2 escorts transforming growth factor α to the basolateral plasma membrane of polarized epithelial cells. Proc. Natl. Acad. Sci. U.S.A. 101, 5571-5576 (2004).
Lovisa, S. et al. Epithelial-to-mesenchymal transition induces cell cycle arrest and parenchymal damage in renal fibrosis. Nat. Med. 21, 998-1009 (2015).
Muto, Y., Wilson, P. C., Wu, H., Waikar, S. S. & Humphreys, B. Single cell transcriptional and chromatin accessibility profiling redefine cellular heterogeneity in the adult human kidney. bioRxiv (2020).
Naba, A. et al. The extracellular matrix: Tools and insights for the ‘omics’ era. Matrix Biol. 49, 10-24 (2016).
Palumbo-Zerr, K. et al. Orphan nuclear receptor NR4A1 regulates transforming growth factor-β signaling and fibrosis. Nat. Med. 21, 150-158 (2015).
Park, J. et al. Single-cell transcriptomics of the mouse kidney reveals potential cellular targets of kidney disease. Science 360, 758-763 (2018).
Picelli, S. et al. Full-length RNA-seq from single cells using Smart-seq2. Nat. Protoc. 9, 171-181 (2014).
Pruenster, M. et al. The Duffy antigen receptor for chemokines transports chemokines and supports their promigratory activity. Nat. Immunol. 10, 101-108 (2009).
Ramachandran, P. et al. Resolving the fibrotic niche of human liver cirrhosis at single-cell level. Nature 575, 512-518 (2019).
Rousset R. et al. Naked cuticle targets dishevelled to antagonize Wnt signal transduction. Genes & development. 15, 658-671 (2001).
Smeets, B. et al. Proximal tubular cells contain a phenotypically distinct, scattered cell population involved in tubular regeneration. J. Pathol. 229, 645-659 (2013).
Stewart, B. J. et al. Spatiotemporal immune zonation of the human kidney. Science 365, 1461-1466 (2019).
Venkatachalam, M. A., Weinberg, J. M., Kriz, W. & Bidani, A. K. Failed Tubule Recovery, AKI-CKD Transition, and Kidney Disease Progression. J. Am. Soc. Nephrol. 26, 1765-1776 (2015).
Wang, Y.-Y. et al. Macrophage-to-Myofibroblast Transition Contributes to Interstitial Fibrosis in Chronic Renal Allograft Injury. J. Am. Soc. Nephrol. 28, 2053-2067 (2017).
Washko, G. R., Homer, R., Yan, X., Rosas, I. O. & Kaminski, N. Single Cell RNA-seq reveals ectopic and aberrant lung resident cell populations in Idiopathic Pulmonary Fibrosis. BioRxiv (2019).
Wernig, G. et al. Unifying mechanism for different fibrotic diseases. Proc. Natl. Acad. Sci. U.S.A. 114, 4757-4762 (2017).
Wilson, P. C. et al. The Single Cell Transcriptomic Landscape of Early Human Diabetic Nephropathy. doi: 10.1101/645424.
Wu, H. et al. Single-Cell Transcriptomics of a Human Kidney Allograft Biopsy Specimen Defines a Diverse Inflammatory Response. J. Am. Soc. Nephrol. (2018) doi: 10.1681/ASN.2018020125.
Wu, H. et al. Comparative Analysis and Refinement of Human PSC-Derived Kidney Organoid Differentiation with Single-Cell Transcriptomics. Cell Stem Cell 23, 869-881.e8 (2018).
Wu, H., Kirita, Y., Donnelly, E. L. & Humphreys, B. D. Advantages of single-nucleus over single-cell RNA sequencing of adult kidney: Rare cell types and novel cell states revealed in fibrosis. J. Am. Soc. Nephrol. 30, 23-32 (2019).
Young, M. D. et al. Single-cell transcriptomes from human kidneys reveal the cellular identity of renal tumors. Science 361, 594-599 (2018).
Zhao, S. et al. NKD2, a negative regulator of Wnt signaling, suppresses tumor growth and metastasis in osteosarcoma. Oncogene 34, 5069-5079 (2015).
Zeng W. et al. Naked cuticle encodes an inducible antagonist of Wnt signalling. Nature. 403, 789-795 (2000).

1. A method for reducing extracellular matrix (ECM) protein expression and/or secretion by a given cell, wherein the method comprises at least one step selected from the group consisting of
(i) inhibiting or reducing nkd2 gene expression in said cell,
(ii) promoting degradation of NKD2 protein in said cell, and/or
(iii) inhibiting or reducing NKD2 protein activity in said cell.

2. The method according to claim 1, wherein the inhibition or reduction of nkd2 gene expression is achieved by nkd2 gene knock-down, knock-out, conditional gene knock-out, gene alteration, RNA interference, siRNA and/or antisense RNA.

3. The method according to claim 1, wherein the inhibition or reduction of NKD2 protein activity is achieved by use of an agent that binds to Naked Cuticle Homolog 2 (NKD2) protein.

4. The method according to claim 1, wherein said cell is a kidney cell, preferably a kidney myofibroblast cell, most preferably a terminally differentiated kidney myofibroblast cell.

5. A method for the identification of an agent that binds to Naked Cuticle Homolog 2 (NKD2) protein, or a fragment thereof.

6. The method according to claim 5, comprising at least the steps of
(i) providing the NKD2 protein, or a fragment thereof,
(ii) adding at least one agent to be screened for binding to the NKD2 protein, or a fragment thereof, and
(iii) identifying the at least one agent that has bound to the NKD2 protein, or the fragment thereof.

7. The method according to claim 5, wherein said agent is an NKD2 inhibitor.

8. The method according to claim 5, wherein said agent is selected from the group consisting of a small-molecule compound, a peptide, and a biologic.

9. The method according to claim 8, wherein said biologic is an antibody, or antigen-binding fragment thereof, or antigen-binding derivative thereof, or antibody-like protein, or an aptamer.

10. The method according to claim 5, wherein the NKD2 protein is bound to a solid phase or is in solution.

11. The method according to claim 6, wherein said agent is member of a compound library.

12. The method according to claim 11, wherein said compound library is comprising small-molecule compounds, peptides, or biologic compounds, respectively.

13. Use of a nucleic acid encoding the naked cuticle homolog 2, or a fragment thereof, or the Naked Cuticle Homolog 2 (NKD2) protein, or a fragment thereof, in a method for the identification of an agent binding to NKD2, or a fragment thereof, according to claim 5.

14. Antibody, or antigen-binding fragment or derivative thereof, or antibody-like protein, that specifically binds to NKD2 protein.

15. Antibody, or antigen-binding fragment or derivative thereof, or antibody-like protein, according to claim 14, wherein said antibody, or antigen-binding fragment or derivative thereof, or antibody-like protein, inhibits the NKD2 activity.

16. An agent obtained by the method according to claim 5.

17. An agent according to claim 16 for use in the treatment of chronic kidney disease.

18. An agent that binds to Naked Cuticle Homolog 2 (NKD2) protein, for use in the treatment of chronic kidney disease.

19. The agent according to claim 18, wherein said agent, when bound to NKD2, inhibits the NKD2 activity.

20. An agent for use according to claim 16, wherein said chronic kidney disease is progressive chronic kidney disease and/or kidney fibrosis.

21. The agent according to claim 16, wherein said agent is a small-molecule compound (smol), a peptide, or a biologic, preferably wherein said biologic is an antibody, or fragment thereof or derivative thereof, or antibody-like protein, or an aptamer.

22. Use of an agent that binds to Naked Cuticle Homolog 2 (NKD2) protein in a method of treating chronic kidney disease, preferably wherein the chronic kidney disease is progressive chronic kidney disease and/or kidney fibrosis.

23. Use of an agent according to claim 22, wherein said agent, when bound to NKD2, inhibits the NKD2 activity.

24. Method for treating or preventing chronic kidney disease, which method comprises administration, to a human or animal subject, of an agent that binds to and/or inhibits Naked Cuticle Homolog 2 (NKD2) protein in a therapeutically effective dose.

25. Pharmaceutical composition comprising the antibody, or antigen-binding fragment or derivative thereof, or antibody-like protein, according to claim 14, and optionally one or more pharmaceutically acceptable excipients.

26. Pharmaceutical composition according to claim 25, wherein said excipients are selected from the group consisting of pharmaceutically acceptable buffers, surfactants, diluents, carriers, excipients, fillers, binders, lubricants, glidants, disintegrants, adsorbents, and/or preservatives.

27. A method for the production of a pharmaceutical composition, comprising
(i) the method according to claim 5, and furthermore
(ii) mixing the agent identified with a pharmaceutically acceptable carrier.

28. A composition comprising a combination of (i) the antibody, or antigen-binding fragment or derivative thereof, or antibody-like protein, according to claim 14, and (ii) one or more further therapeutically active compounds.

29. A therapeutic kit of parts comprising:
(i) the pharmaceutical composition according to claim 25,
(ii) a device for administering the composition, and
(iii) optionally instructions for use.


SEQUENCE LISTING (1 sequence)

SEQ ID NO: 1 - protein, 451 aa, NKD2-Protein, human
  NKD2-Protein, human
MGKLQSKHAAAARKRRESPEGDSFVASAYASGRKGAEEAERRARDKQELPNGDPKEGPFREDQCPLQVALPAEKAEGREHPGQLLSADDGERAANREGPRGPGGQRLNIDALQCDVSVEEDDRQEWTFTLYDFDNCGKVTREDMSSLMHTIYEVVDASVNHSSGSSKTLRVKLTVSPEPSSKRKEGPPAGQDREPTRCRMEGELAEEPRVADRRLSAHVRRPSTDPQPCSERGPYCVDENTERRNHYLDLAGIENYTSRFGPGSPPVQAKQEPQGRASHLQARSRSQEPDTHAVHHRRSQVLVEHVVPASEPAARALDTQPRPKGPEKQFLKSPKGSGKPPGVPASSKSGKAFSYYLPAVLPPQAPQDGHHLPQPPPPPYGHKRYRQKGREGHSPLKAPHAQPATVEHEVVRDLPPTPAGEGYAVPVIQRHEHHHHHEHHHHHHHHHFHPS